(19) United States
(12) Patent Application Publication
(10) Pub. No.: US 2018/0147196 A1
(43) Pub. Date: May 31, 2018

(54) PYRIDIN-3-YL ACETIC ACID DERIVATIVES AS INHIBITORS OF HUMAN IMMUNODEFICIENCY VIRUS REPLICATION

(71) Applicant: ViiV HEALTHCARE UK (NO.5) LIMITED, Brentford, Middlesex (GB)

(72) Inventors: Kyle J. EASTMAN, Wallingford, CT (US); John F. KADOW, Wallingford, CT (US); B. Narasimhulu NAIDU, Wallingford, CT (US); Kyle E. PARCELLA, Wallingford, CT (US); Manoj PATEL, Wallingford, CT (US); Yong TU, Wallingford, CT (US)

(21) Appl. No.: 15/580,742

(22) PCT Filed: Jul. 7, 2016
(86) PCT No.: PCT/IB2016/054089

§ 371 Date: Dec. 8, 2017

Related U.S. Application Data
(60) Provisional application No. 62/190,598, filed on Jul. 9, 2015.

(51) Int. Cl. A61K 31/4545; C07D 401/04; C07D 401/14; A61K 31/5365; A61P 31/18

(52) U.S. Cl. CPC A61K 31/4545; C07D 401/04; A61P 31/18; A61K 31/5365; C07D 401/14

(57) ABSTRACT

Disclosed are compounds of Formula I, including pharmaceutically acceptable salts, pharmaceutical compositions comprising the compounds, methods for making the compounds and their use in inhibiting HIV integrase and treating those infected with HIV or AIDS.

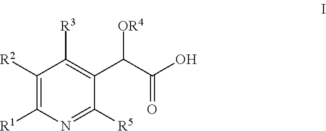

### CROSS REFERENCE TO RELATED INVENTION

This application claims the benefit of U.S. provisional application Ser. No. 62/190,598 filed Jul. 8, 2015.

### FIELD OF THE INVENTION

The invention relates to compounds, compositions, and methods for the treatment of human immunodeficiency virus (HIV) infection. More particularly, the invention provides novel inhibitors of HIV, pharmaceutical compositions containing such compounds, and methods for using these compounds in the treatment of HIV infection. The invention also relates to methods for making the compounds hereinafter described.

### BACKGROUND OF THE INVENTION

Human immunodeficiency virus (HIV) has been identified as the etiological agent responsible for acquired immune deficiency syndrome (AIDS), a fatal disease characterized by destruction of the immune system and the inability to fight off life threatening opportunistic infections. Recent statistics indicate that an estimated 35.3 million people worldwide are infected with the virus (UNAIDS: Report on the Global HIV/AIDS Epidemic, 2013). In addition to the large number of individuals already infected, the virus continues to spread. Estimates from 2013 point to close to 3.4 million new infections in that year alone. In the same year there were approximately 1.6 million deaths associated with HIV and AIDS.

Current therapy for HIV-infected individuals consists of a combination of approved anti-retroviral agents. Over two dozen drugs are currently approved for HIV infection, either as single agents or as fixed dose combinations or single tablet regimens, the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIs), integrase inhibitors (INIs), or entry inhibitors (one, maraviroc, targets the host CCRS protein, while the other, enfuvirtide, is a peptide that targets the gp41 region of the viral gp160 protein). In addition, a pharmacokinetic enhancer with no antiviral activity, i.e., cobicistat, available from Gilead Sciences, Inc. under the tradename TYBOST™ (cobicistat) tablets, has recently been approved for use in combinations with certain antiretroviral agents (ARVs) that may benefit from boosting.

In the US, where combination therapy is widely available, the number of HIV-related deaths has dramatically declined (Palella, F. J.; Delany, K. M.; Moorman, A. C.; Loveless, M. O.; Furher, J.; Satten, G. A.; Aschman, D. J.; Holmberg, S. D. N. Engl. J. Med. 1998, 338, 853-860).

Unfortunately, not all patients are responsive and a large number fail this therapy. In fact, initial studies suggest that approximately 30-50% of patients ultimately fail at least one drug in the suppressive combination. Treatment failure in most cases is caused by the emergence of viral resistance. Viral resistance in turn is caused by the replication rate of HIV-1 during the course of infection combined with the relatively high viral mutation rate associated with the viral polymerase and the lack of adherence of HIV-infected individuals in taking their prescribed medications. Clearly, there is a need for new antiviral agents, preferably with activity against viruses already resistant to currently approved drugs. Other important factors include improved safety and a more convenient dosing regimen than many of the currently approved drugs.

Compounds which inhibit HIV replication have been disclosed. See, for example, the following patent applications: WO2007131350, WO2009062285, WO2009062288, WO2009062289, WO2009062308, WO2010130034, WO2010130842, WO2011015641, WO2011076765, WO2012033735, WO2013123148, WO2013134113, WO2014164467, WO2014159959, and WO2015126726.

What is now needed in the art are additional compounds which are novel and useful in the treatment of HIV. Additionally, these compounds may desireably provide advantages for pharmaceutical uses, for example, with regard to one or more of their mechanisms of action, binding, inhibition efficacy, target selectivity, solubility, safety profiles, or bioavailability. Also needed are new formulations and methods of treatment which utilize these compounds.

### SUMMARY OF THE INVENTION

The invention encompasses compounds of Formula I, including pharmaceutically acceptable salts thereof, as well as pharmaceutical compositions, and their use in inhibiting HIV and treating those infected with HIV or AIDS.

By virtue of the present invention, it is now possible to provide compounds that are novel and are useful in the treatment of HIV. Additionally, the compounds may provide advantages for pharmaceutical uses, for example, with regard to one or more of their mechanism of action, binding, inhibition efficacy, target selectivity, solubility, safety profiles, or bioavailability.

The invention also provides pharmaceutical compositions comprising the compounds of the invention, including pharmaceutically acceptable salts thereof, and a pharmaceutically acceptable carrier, excipient, and/or diluent.

In addition, the invention provides methods of treating HIV infection comprising administering a therapeutically effective amount of the compounds of the invention to a patient.

In addition, the invention provides methods for inhibiting HIV integrase.

Also provided in accordance with the invention are methods for making the compounds of the invention.

The present invention is directed to these, as well as other important ends, hereinafter described.

### DESCRIPTION OF THE INVENTION

Unless specified otherwise, these terms have the following meanings.

“Alkyl” means a straight or branched saturated hydrocarbon comprised of 1 to 10 carbons, and preferably 1 to 6 carbons.

“Alkenyl” means a straight or branched alkyl group comprised of 2 to 10 carbons with at least one double bond and optionally substituted with 0-3 halo or alkoxy group.

“Alkynyl” means a straight or branched alkyl group comprised of 2 to 10 carbons, preferably 2 to 6 carbons, containing at least one triple bond and optionally substituted with 0-3 halo or alkoxy group.

“Aryl” mean a carbocyclic group comprised of 1-3 rings that are fused and/or bonded and at least one or a combination of which is aromatic. The non-aromatic carbocyclic portion, where present, will be comprised of C₃ to C₇ alkyl group. Examples of aromatic groups include, but are not limited to indanyl, indenyl, naphthyl, phenyl, tetrahydronaphthyl and cyclopropylphenyl. The aryl group can be attached to the parent structure through any substitutable carbon atom in the group.

“Arylalkyl” is a C₁-C₅ alkyl group attached to 1 to 2 aryl groups and linked to the parent structure through the alkyl moiety. Examples include, but are not limited to, —(CH₂)ₙPh with n=1-5, —CH(CH₃)Ph, —CH(Ph)₂.

“Aryloxy” is an aryl group attached to the parent structure by oxygen.

“Cycloalkyl” means a monocyclic ring system composed of 3 to 7 carbons.

“Halo” includes fluoro, chloro, bromo, and iodo.

“Haloalkyl” and “haloalkoxy” include all halogenated isomers from monohalo to perhalo.

“Heteroaryl” is a subset of heterocyclic group as defined below and is comprised of 1-3 rings where at least one or a combination of which is aromatic and that the aromatic group contains at least one atom chosen from a group of oxygen, nitrogen or sulfur.

“Heterocyclyl or heterocyclic” means a cyclic group of 1-3 rings comprised of carbon and at least one other atom selected independently from oxygen, nitrogen and sulfur. The rings could be bridged, fused and/or bonded, through a direct or spiro attachment, with the option to have one or a combination thereof be aromatic. Examples include, but are not limited to, azaindole, azaindoline, azetidine, benzimidazole, bezodioxolyl, benzoisothiazole, benzothiazole, benzothiadiazole, benzothiophene, benzoxazole, carbazole, chroman, dihalobezodioxolyl, dihydrobenzofuran, dihydro-benzo[1,4]oxazine, 1,3-dihydrobenzo[c]thiophene 2,2-dioxide, 2,3-dihydrobenzo[d]isothiazole 1,1-dioxide, 3,4-dihydro-2H-pyrido[3,2-b][1,4]oxazine, 2,3-dihydro-1H-pyrrolo[3,4-c]pyridine and its regioisomeric variants, 6,7-dihydro-5H-pyrrolo[2,3-b]pyrazine and its regioisomeric variants, furanylphenyl, imidazole, imidazo[1,2-a]pyridine, indazole, indole, indoline, isoquinoline, isoquinolinone, isothiazolidine 1,1-dioxide, morpholine, 2-oxa-5-azabicyclo[2.2.1]heptane, oxadiazole-phenyl, oxazole, phenylaztidine, phenylindazole, phenylpiperidine, phenylpiperizine, phenyloxazole, phenylpyrrolidine, piperidine, pyridine, pyridinylphenyl, pyridinylpyrrolidine, pyrimidine, pyrimidinylphenyl, pyrrazole-phenyl, pyrrolidine, pyrrolidin-2-one, 1H-pyrazolo[4,3-c]pyridine and its regioisomeric variants, pyrrole, 5H-pyrrolo[2,3-b]pyrazine, 7H-pyrrolo[2,3-d]pyrimidine and its regioisomeric variants, quinazoline, quinoline, quinoxaline, tetrahydroisoquinoline, 1,2,3,4-tetrahydro-1,8-naphthyridine, tetrahydroquinoline, 4,5,6,7-tetrahydrothieno[3,2-c]pyridine, 1,2,5-thiadiazolidine 1,1-dioxide, thiophene, thiophenylphenyl, triazole, or triazolone. Unless otherwise specifically set forth, the heterocyclic group can be attached to the parent structure through any suitable atom in the group that results in a stable compound.

It is understood that a subset of the noted heterocyclic examples encompass regioisomers. For instance, “azaindole” refers to any of the following regioisomers: 1H-pyrrolo[2,3-b]pyridine, 1H-pyrrolo[2,3-c]pyridine, 1H-pyrrolo[3,2-c]pyridine, and 1H-pyrrolo[3,2-b]pyridine. In addition the “regioisomer variants” notation as in, for example, “5H-pyrrolo[2,3-b]pyrazine and its regioisomeric variants” would also encompass 7H-pyrrolo[2,3-d]pyrimidine, 7H-pyrrolo[2,3-c]pyridazine, 1H-pyrrolo[2,3-d]pyridazine, 5H-pyrrolo[3,2-c]pyridazine, and 5H-pyrrolo[3,2-d]pyrimidine. Similarly, 6,7-dihydro-5H-pyrrolo[2,3-b]pyrazine and its regioisomeric variants would encompass 6,7-dihydro-5H-pyrrolo[2,3-d]pyrimidine and 6,7-dihydro-5H-pyrrolo[2,3-c]pyridazine. It is also understood that the lack of “regioisomeric variants” notation does not in any way restrict the claim scope to the noted example only.

“Heterocyclylalkyl” is a heterocyclyl moiety attached to the parent structure through C₁-C₅ alkyl group. Examples include, but are not limited to, —(CH₂)ₙ—R^{Z} or —CH(CH₃)—(R^{Z}) where n=1-5 and that R^{Z} is chosen from benzimidazole, imidazole, indazole, isooxazole, phenyl-pyrazole, pyridine, quinoline, thiazole, triazole, triazolone, oxadiazole.

Terms with a hydrocarbon moiety (e.g. alkoxy) include straight and branched isomers for the hydrocarbon portion with the indicated number of carbon atoms.

Bonding and positional bonding relationships are those that are stable as understood by practitioners of organic chemistry.

Parenthetic and multiparenthetic terms are intended to clarify bonding relationships to those skilled in the art. For example, a term such as ((R)alkyl) means an alkyl substituent further substituted with the substituent R.

Substituents which are illustrated by chemical drawing to bond at variable positions on a multiple ring system (for example a bicyclic ring system) are intended to bond to the ring where they are drawn to append. Parenthetic and multiparenthetic terms are intended to clarify bonding relationships to those skilled in the art. For example, a term such as ((R)alkyl) means an alkyl substituent further substituted with the substituent R.

“Combination,” “coadministration,” “concurrent” and similar terms referring to the administration of a compound of Formula I with at least one anti-HIV agent mean that the components are part of a combination antiretroviral therapy or highly active antiretroviral therapy (“HAART”) as understood by practitioners in the field of AIDS and HIV infection.

“Therapeutically effective” means the amount of agent required to provide a benefit to a patient as understood by practitioners in the field of AIDS and HIV infection. In general, the goals of treatment are suppression of viral load, restoration and preservation of immunologic function, improved quality of life, and reduction of HIV-related morbidity and mortality.

“Patient” means a person infected with the HIV virus.

“Treatment,” “therapy,” “regimen,” “HIV infection,” “ARC,” “AIDS” and related terms are used as understood by practitioners in the field of AIDS and HIV infection.

Those terms not specifically set forth herein shall have the meaning which is commonly understood and accepted in the art.

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Some of the compounds of the invention exist in stereoisomeric forms. The invention includes all stereoisomeric forms of the compounds including enantiomers and diastereromers. Methods of making and separating stereoisomers are known in the art. The invention includes all tautomeric forms of the compounds. The invention includes atropisomers and rotational isomers.

The invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

In an aspect of the invention, there is provided a compound of Formula I:

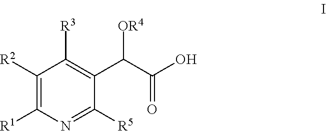

wherein:
R¹ is selected from hydrogen or alkyl;
R² is selected from hydrogen, halo, cyano, alkyl, (R⁶)alkyl, alkenyl, (R⁶)alkenyl, alkynyl, (R⁶)alkynyl, cycloalkyl, (alkyl)cycloalkyl, cycloalkenyl, (alkyl)cycloalkenyl, (R⁶)cycloalkenyl, (R⁷)NHCH₂CH═CH—, (R⁷)tetrahydropyridinyl, or ((N-benzyl-4-hydroxy)piperidin-4-yl)ethynyl;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, alkoxy, and haloalkoxy;
R⁴ is selected from alkyl or haloalkyl;
R⁵ is alkyl;
R⁶ is selected from Ar¹, (Ar¹)alkyl, (Ar¹O)alkyl or benzyloxy,
R⁷ is selected from hydrogen, (Ar¹)alkyl, alkoxycarbonyl, or benzyloxycarbonyl; and
Ar¹ is phenyl substituted with 0-3 substituents selected from halo, alkyl, haloalkyl, alkoxy, haloalkoxy, or phenyl;
or a pharmaceutically acceptable salt thereof.

In an aspect of the invention, R³ is piperidinyl substituted with 0-3 substituents selected from cyano, halo, alkyl, haloalkyl, alkoxy, or haloalkoxy. In an aspect of the invention, R² is selected from alkyl, (R⁶)alkyl, alkenyl, (R⁶)alkenyl, alkynyl, or (R⁶)alkynyl. In an aspect of the invention, R² is selected from cycloalkyl, (alkyl)cycloalkyl, cycloalkenyl, (alkyl)cycloalkenyl, or (R⁶)cycloalkenyl. In an aspect of the invention, R² is (R⁷)NHCH₂CH═CH— or (R⁷)tetrahydropyridinyl.

In an aspect of the invention, there is provided a compound of Formula I:

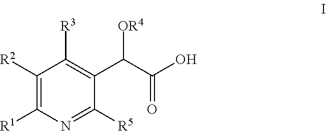

wherein:
R¹ is selected from hydrogen or alkyl;
R² is selected from hydrogen, halo, cyano, alkyl, (R⁶)alkyl, alkenyl, (R⁶)alkenyl, alkynyl, (R⁶)alkynyl, cycloalkyl, (alkyl)cycloalkyl, cycloalkenyl, (alkyl)cycloalkenyl, (R⁶)cycloalkenyl, (R⁷)NHCH₂CH═CH—, (R⁷)tetrahydropyridinyl, or ((N-benzyl-4-hydroxy)piperidin-4-yl)ethynyl;
R³ is piperidinyl substituted with 0-3 substituents selected from cyano, halo, alkyl, haloalkyl, alkoxy, or haloalkoxy;
R⁴ is selected from alkyl or haloalkyl;
R⁵ is alkyl;
R⁶ is selected from Ar¹, (Ar¹)alkyl, (Ar¹O)alkyl or benzyloxy,
R⁷ is selected from hydrogen, (Ar¹)alkyl, alkoxycarbonyl, or benzyloxycarbonyl; and
Ar¹ is phenyl substituted with 0-3 substituents selected from halo, alkyl, haloalkyl, alkoxy, haloalkoxy, or phenyl;
or a pharmaceutically acceptable salt thereof.

In an aspect of the invention, there is provided a compound of Formula I:

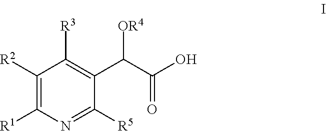

wherein:
R¹ is selected from hydrogen or alkyl;
R² is selected from alkyl, (R⁶)alkyl, alkenyl, (R⁶)alkenyl, alkynyl, or (R⁶)alkynyl;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, alkoxy, and haloalkoxy;
R⁴ is selected from alkyl or haloalkyl;
R⁵ is alkyl;
R⁶ is selected from Ar¹, (Ar¹)alkyl, (Ar¹O)alkyl or benzyloxy,
R⁷ is selected from hydrogen, (Ar¹)alkyl, alkoxycarbonyl, or benzyloxycarbonyl; and
Ar¹ is phenyl substituted with 0-3 substituents selected from halo, alkyl, haloalkyl, alkoxy, haloalkoxy, or phenyl;
or a pharmaceutically acceptable salt thereof.

In an aspect of the invention, there is provided a compound of Formula I:

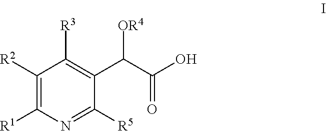

wherein:
R¹ is selected from hydrogen or alkyl;
R² is selected from cycloalkyl, (alkyl)cycloalkyl, cycloalkenyl, (alkyl)cycloalkenyl, or (R⁶)cycloalkenyl;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, alkoxy, and haloalkoxy;
R⁴ is selected from alkyl or haloalkyl;
R⁵ is alkyl;
R⁶ is selected from Ar¹, (Ar¹)alkyl, (Ar¹O)alkyl or benzyloxy,
R⁷ is selected from hydrogen, (Ar¹)alkyl, alkoxycarbonyl, or benzyloxycarbonyl; and
Ar¹ is phenyl substituted with 0-3 substituents selected from halo, alkyl, haloalkyl, alkoxy, haloalkoxy, or phenyl;
or a pharmaceutically acceptable salt thereof.

In an aspect of the invention, there is provided a compound of Formula I:

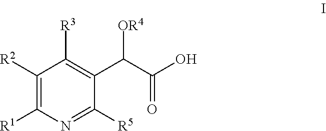

wherein:
R¹ is selected from hydrogen or alkyl;
R² is selected from (R⁷)NHCH₂CH═CH— or (R⁷)tetrahydropyridinyl;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, alkoxy, and haloalkoxy;
R⁴ is selected from alkyl or haloalkyl;
R⁵ is alkyl;
R⁶ is selected from Ar¹, (Ar¹)alkyl, (Ar¹O)alkyl or benzyloxy,
R⁷ is selected from hydrogen, (Ar¹)alkyl, alkoxycarbonyl, or benzyloxycarbonyl; and
Ar¹ is phenyl substituted with 0-3 substituents selected from halo, alkyl, haloalkyl, alkoxy, haloalkoxy, or phenyl;
or a pharmaceutically acceptable salt thereof.

For a particular compound of Formula I, the scope of any instance of a variable substituent, including R¹, R², R³, R⁴, R⁵, R⁶, R⁷ and Ar¹ can be used independently with the scope of any other instance of a variable substituent. As such, the invention includes combinations of the different aspects.

In an aspect of the invention, there is provided a composition useful for treating HIV infection comprising a therapeutic amount of a compound of Formula I and a pharmaceutically acceptable carrier. In an aspect of the invention, the composition further comprises a therapeutically effective amount at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier. In an aspect of the invention, the other agent is dolutegravir.

In an aspect of the invention, there is provided a method for treating HIV infection comprising administering a therapeutically effective amount of a compound of Formula I, or a pharmaceutically acceptable salt thereof, to a patient in need thereof. In an aspect of the invention, the method further comprises administering a therapeutically effective amount of at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors. In an aspect of the invention, the other agent is dolutegravir. In an aspect of the invention, the other agent is administered to the patient prior to, simultaneously with, or subsequently to the compound of Formula I.

Preferred compounds in accordance with the present invention include the following:
(S)-2-(5-(3-(Benzyloxy)prop-1-yn-1-yl)-4-(4,4-dimethylpiperidin-1-yl)-2,6-dimethylpyridin-3-yl)-2-(tert-butoxy)acetic acid;
(S)-2-(5-((1-Benzyl-4-hydroxypiperidin-4-yl)ethynyl)-4-(4,4-dimethylpiperidin-1-yl)-2,6-dimethylpyridin-3-yl)-2-(tert-butoxy)acetic acid;
(S,E)-2-(tert-Butoxy)-2-(4-(4,4-dimethylpiperidin-1-yl)-5-(4-fluorostyryl)-2,6-dimethylpyridin-3-yl)acetic acid;
(S)-2-(tert-Butoxy)-2-(4-(4,4-dimethylpiperidin-1-yl)-5-(4-fluorophenethyl)-2,6-dimethylpyridin-3-yl)acetic acid;
(S,E)-2-(5-(2-([1,1′-Biphenyl]-4-yl)vinyl)-4-(4,4-dimethylpiperidin-1-yl)-2,6-dimethylpyridin-3-yl)-2-(tert-butoxy)acetic acid;
(S)-2-(5-(2-([1,1′-Biphenyl]-4-yl)ethyl)-4-(4,4-dimethylpiperidin-1-yl)-2,6-dimethylpyridin-3-yl)-2-(tert-butoxy)acetic acid;
(S,E)-2-(tert-Butoxy)-2-(4-(4,4-dimethylpiperidin-1-yl)-2,6-dimethyl-5-(4-(trifluoromethyl)styryl)pyridin-3-yl)acetic acid;
(S)-2-(tert-Butoxy)-2-(4-(4,4-dimethylpiperidin-1-yl)-2,6-dimethyl-5-(4-(trifluoromethyl)phenethyl)pyridin-3-yl)acetic acid;
(S,E)-2-(tert-Butoxy)-2-(4-(4,4-dimethylpiperidin-1-yl)-2,6-dimethyl-5-(3-phenylprop-1-en-1-yl)pyridin-3-yl)acetic acid;
(S)-2-(tert-Butoxy)-2-(4-(4,4-dimethylpiperidin-1-yl)-2,6-dimethyl-5-(3-phenylpropyl)pyridin-3-yl)acetic acid;
(S,E)-2-(tert-Butoxy)-2-(4-(4,4-dimethylpiperidin-1-yl)-2,6-dimethyl-5-styrylpyridin-3-yl)acetic acid;
(S)-2-(tert-Butoxy)-2-(4-(4,4-dimethylpiperidin-1-yl)-2,6-dimethyl-5-phenethylpyridin-3-yl)acetic acid;
(S)-2-(5-Bromo-4-(4,4-dimethylpiperidin-1-yl)-2,6-dimethylpyridin-3-yl)-2-(tert-butoxy)acetic acid;
(S)-2-(tert-Butoxy)-2-(5-(cyclohex-1-en-1-yl)-4-(4,4-dimethylpiperidin-1-yl)-2,6-dimethylpyridin-3-yl)acetic acid;
(2S)-2-(tert-Butoxy)-2-(5-(4-(tert-butyl)cyclohex-1-en-1-yl)-4-(4,4-dimethylpiperidin-1-yl)-2,6-dimethylpyridin-3-yl)acetic acid;
(S)-2-(tert-Butoxy)-2-(1′-(tert-butoxycarbonyl)-4-(4,4-dimethylpiperidin-1-yl)-2,6-dimethyl-1′,2′,3′,6′-tetrahydro-[3,4′-bipyridin]-5-yl)acetic acid;
(S,E)-2-(5-(3-(((Benzyloxy)carbonyl)amino)prop-1-en-1-yl)-4-(4,4-dimethylpiperidin-1-yl)-2,6-dimethylpyridin-3-yl)-2-(tert-butoxy)acetic acid;
(S,E)-2-(tert-Butoxy)-2-(4-(4,4-dimethylpiperidin-1-yl)-5-(3-((methoxycarbonyl)amino)prop-1-en-1-yl)-2,6-dimethylpyridin-3-yl)acetic acid;
(S)-2-(1′-((Benzyloxy)carbonyl)-4-(4,4-dimethylpiperidin-1-yl)-2,6-dimethyl-1′,2′,3′,6′-tetrahydro-[3,4′-bipyridin]-5-yl)-2-(tert-butoxy)acetic acid;
(S)-2-(tert-Butoxy)-2-(4-(4,4-dimethylpiperidin-1-yl)-1′-(methoxycarbonyl)-2,6-dimethyl-1′,2′,3′,6′-tetrahydro-[3,4′-bipyridin]-5-yl)acetic acid;
(S)-2-(tert-Butoxy)-2-(4-(4,4-dimethylpiperidin-1-yl)-2,6-dimethyl-1′,2′,3′,6′-tetrahydro-[3,4′-bipyridin]-5-yl)acetic acid;
(S)-2-(tert-Butoxy)-2-(4-(4,4-dimethylpiperidin-1-yl)-1′-(4-fluorophenethyl)-2,6-dimethyl-1′,2′,3′,6′-tetrahydro-[3,4′-bipyridin]-5-yl)acetic acid;
(S)-2-(tert-Butoxy)-2-(4-(4,4-dimethylpiperidin-1-yl)-1′-(3-(4-fluorophenyl)propyl)-2,6-dimethyl-1′,2′,3′,6′-tetrahydro-[3,4′-bipyridin]-5-yl)acetic acid;
(S)-2-(tert-butoxy)-2-(5-cyano-4-(4,4-dimethylpiperidin-1-yl)-2,6-dimethylpyridin-3-yl)acetic acid;
(2S)-2-(tert-butoxy)-2-(4-(4,4-dimethylpiperidin-1-yl)-5-(3-(2-(4-fluorophenoxy)ethyl)cyclobut-1-en-1-yl)-2,6-dimethylpyridin-3-yl)acetic acid;
(S)-2-(tert-Butoxy)-2-(4-(4,4-dimethylpiperidin-1-yl)-2,5,6-trimethylpyridin-3-yl)acetic acid;
(S)-2-(tert-Butoxy)-2-(4-(4,4-dimethylpiperidin-1-yl)-2,6-dimethylpyridin-3-yl)acetic acid;
(S)-2-(tert-Butoxy)-2-(4-(4,4-dimethylpiperidin-1-yl)-2,6-dimethyl-5-vinylpyridin-3-yl)acetic acid;
(S)-2-(tert-Butoxy)-2-(4-(4,4-dimethylpiperidin-1-yl)-5-ethyl-2,6-dimethylpyridin-3-yl)acetic acid;
(S,E)-2-(tert-Butoxy)-2-(4-(4,4-dimethylpiperidin-1-yl)-2,6-dimethyl-5-(prop-1-en-1-yl)pyridin-3-yl)acetic acid;
(S)-2-(tert-Butoxy)-2-(4-(4,4-dimethylpiperidin-1-yl)-1′-(3-(4-fluorophenyl)propyl)-6-methyl-1′,2′,3′,6′-tetrahydro-[3,4′-bipyridin]-5-yl)acetic acid; and
(S)-2-(tert-Butoxy)-2-(5-cyclopropyl-4-(4,4-dimethylpiperidin-1-yl)-2,6-dimethylpyridin-3-yl)acetic acid; and
(S)-2-(tert-Butoxy)-2-(4-(4,4-dimethylpiperidin-1-yl)-1′-(3-(4-fluorophenyl)propyl)-6-methyl-1′,2′,3′,6′-tetrahydro-[3,4′-bipyridin]-5-yl)acetic acid; and
pharmaceutically acceptable salts thereof.

The compounds of the invention herein described may typically be administered as pharmaceutical compositions. These compositions are comprised of a therapeutically effective amount of a compound of Formula I or its pharmaceutically acceptable salt, and a pharmaceutically acceptable carrier and may contain conventional excipients and/or diluents. A therapeutically effective amount is that which is needed to provide a meaningful patient benefit. Pharmaceutically acceptable carriers are those conventionally known carriers having acceptable safety profiles. Compositions encompass all common solid and liquid forms, including capsules, tablets, lozenges, and powders, as well as liquid suspensions, syrups, elixirs, and solutions. Compositions are made using available formulation techniques, and excipients (such as binding and wetting agents) and vehicles (such as water and alcohols) which are generally used for compositions. See, for example, Remington's Pharmaceutical Sciences, 17th edition, Mack Publishing Company, Easton, Pa. (1985).

Solid compositions which are normally formulated in dosage units and compositions providing from about 1 to 1000 milligram (“mg”) of the active ingredient per dose are typical. Some examples of dosages are 1 mg, 10 mg, 100 mg, 250 mg, 500 mg, and 1000 mg. Generally, other antiretroviral agents will be present in a unit range similar to agents of that class used clinically. Typically, this is about 0.25-1000 mg/unit.

Liquid compositions are usually in dosage unit ranges. Generally, the liquid composition will be in a unit dosage range of about 1-100 milligram per milliliter (“mg/mL”). Some examples of dosages are 1 mg/mL, 10 mg/mL, 25 mg/mL, 50 mg/mL, and 100 mg/mL. Generally, other antiretroviral agents will be present in a unit range similar to agents of that class used clinically. Typically, this is about 1-100 mg/mL.

The invention encompasses all conventional modes of administration; oral and parenteral methods are preferred. Generally, the dosing regimen will be similar to other antiretroviral agents used clinically. Typically, the daily dose will be about 1-100 milligram per kilogram (“mg/kg”) body weight daily. Generally, more compound is required orally and less parenterally. The specific dosing regimen, however, will be determined by a physician using sound medical judgment.

The compounds of this invention desireably have activity against HIV. Accordingly, another aspect of the invention is a method for treating HIV infection in a human patient comprising administering a therapeutically effective amount of a compound of Formula I, or a pharmaceutically acceptable salt thereof, with a pharmaceutically acceptable carrier, excipient and/or diluent.

The invention also encompasses methods where the compound is given in combination therapy. That is, the compound can be used in conjunction with, but separately from, other agents useful in treating AIDS and HIV infection. The compound can also be used in combination therapy wherein the compound and one or more of the other agents are physically together in a fixed-dose combination (FDC). Some of these agents include HIV attachment inhibitors, CCRS inhibitors, CXCR4 inhibitors, HIV cell fusion inhibitors, HIV integrase inhibitors, HIV nucleoside reverse transcriptase inhibitors, HIV non-nucleoside reverse transcriptase inhibitors, HIV protease inhibitors, budding and maturation inhibitors, HIV capsid inhibitors, anti-infectives, and immunomodulators, such as, for example, PD-1 inhibitors, PD-L1 inhinitors, antibodies, and the like. In these combination methods, the compound of Formula I will generally be given in a daily dose of about 1-100 mg/kg body weight daily in conjunction with other agents. The other agents generally will be given in the amounts used therapeutically. The specific dosing regimen, however, will be determined by a physician using sound medical judgment.

Examples of nucleoside HIV reverse transcriptase inhibitors include abacavir, didanosine, emtricitabine, lamivudine, stavudine, tenofovir, zalcitabine, and zidovudine.

Examples of non-nucleoside HIV reverse transcriptase inhibitors include delavirdine, efavirenz, etrivirine, nevirapine, and rilpivirine.

Examples of HIV protease inhibitors include amprenavir, atazanavir, darunavir, fosamprenavir, indinavir, lopinavir, nelfinavir, ritonavir, saquinavir and, tipranavir.

An example of an HIV fusion inhibitor is enfuvirtide or T-1249.

An example of an HIV entry inhibitor is maraviroc.

Examples of HIV integrase inhibitors include dolutegravir, elvitegravir, or raltegravir.

An example of an HIV attachment inhibitor is fostemsavir.

An example of an HIV maturation inhibitor is BMS-955176, having the following structure:

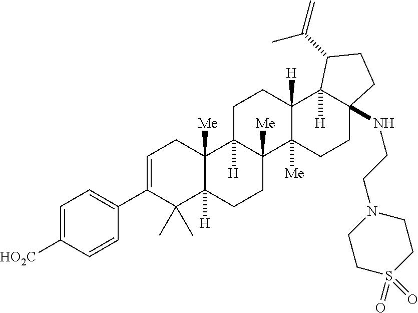

Thus, as set forth above, contemplated herein are combinations of the compounds of Formula I, together with one or more agents useful in the treatment of AIDS. For example, the compounds of the invention may be effectively administered, whether at periods of pre-exposure and/or post-exposure, in combination with effective amounts of the AIDS antivirals, immunomodulators, anti-infectives, or vaccines, such as those in the following non-limiting table:

| | | |
|---|---|---|
| Drug Name | Manufacturer | Indication |

ANTIVIRALS

| | | |
|---|---|---|
| Rilpivirine | Tibotec | HIV infection, AIDS, ARC |
| | | (non-nucleoside reverse |
| | | transcriptase inhibitor) |
| COMPLERA ® | Gilead | HIV infection, AIDS, ARC; |
| | | combination with emtricitabine, |
| | | rilpivirine, and tenofovir disoproxil |
| | | fumarate |
| 097 | Hoechst/Bayer | HIV infection, AIDS, ARC |
| | | (non-nucleoside reverse |
| | | transcriptase (RT) inhibitor) |
| Amprenavir 141 | Glaxo Wellcome | HIV infection, AIDS, ARC |
| W94 GW 141 | | (protease inhibitor) |
| Abacavir (1592U89) | Glaxo Wellcome | HIV infection, AIDS, ARC |
| GW 1592 | | (RT inhibitor) |
| Acemannan | Carrington Labs | ARC |
| | (Irving, TX) | |
| Acyclovir | Burroughs Wellcome | HIV infection, AIDS, ARC |
| AD-439 | Tanox Biosystems | HIV infection, AIDS, ARC |
| AD-519 | Tanox Biosystems | HIV infection, AIDS, ARC |
| Adefovir dipivoxil | Gilead Sciences Ethigen | HIV infection ARC, PGL |
| AL-721 | (Los Angeles, CA) | HIV positive, AIDS |
| Alpha Interferon | Glaxo Wellcome | Kaposi's sarcoma, |
| | | HIV in combination w/Retrovir |
| Ansamycin | Adria Laboratories | ARC |
| LM 427 | (Dublin, OH) | |
| | Erbamont (Stamford, CT) | |
| Antibody which | Advanced Biotherapy | AIDS, ARC |
| Neutralizes pH | Concepts | |
| Labile alpha aberrant | (Rockville, MD) | |
| Interferon | | |
| AR177 | Aronex Pharm | HIV infection, AIDS, ARC |
| Beta-fluoro-ddA | Nat'l Cancer Institute | AIDS-associated diseases |
| CI-1012 | Warner-Lambert | HIV-1 infection |
| Cidofovir | Gilead Science | CMV retinitis, |
| | | herpes, papillomavirus |
| Curdlan sulfate | AJI Pharma USA | HIV infection |
| Cytomegalovirus | MedImmune | CMV retinitis |
| Immune globin | | |
| Cytovene | Syntex | Sight threatening |
| Ganciclovir | | CMV peripheral |
| | | CMV retinitis |
| Darunavir | Tibotec- J & J | HIV infection, AIDS, ARC |
| | | (protease inhibitor) |
| Delaviridine | Pharmacia-Upjohn | HIV infection, AIDS, ARC |
| | | (RT inhibitor) |
| Dextran Sulfate | Ueno Fine Chem. Ind. | AIDS, ARC, HIV positive |
| | Ltd. (Osaka, Japan) | asymptomatic |
| ddC | Hoffman-La Roche | HIV infection, AIDS, ARC |
| Dideoxycytidine | | |
| ddI | Bristol-Myers Squibb | HIV infection, AIDS, ARC; |
| Dideoxyinosine | | combination with AZT/d4T |
| DMP-450 | AVID | HIV infection, AIDS, ARC |
| | (Camden, NJ) | (protease inhibitor) |
| Efavirenz | Bristol Myers Squibb | HIV infection, AIDS, ARC |
| (DMP 266, SUSTIVA ®) | | (non-nucleoside RT inhibitor) |
| (−)6-Chloro-4-(S)- | | |
| cyclopropylethynyl- | | |
| 4(S)-trifluoromethyl- | | |
| 1,4-dihydro-2H-3,1- | | |
| benzoxazin-2-one, STOCRINE | | |
| EL10 | Elan Corp, PLC | HIV infection |
| | (Gainesville, GA) | |
| Etravirine | Tibotec/J & J | HIV infection, AIDS, ARC |
| | | (non-nucleoside reverse |
| | | transcriptase inhibitor) |
| Famciclovir | Smith Kline | herpes zoster, herpes simplex |
| GS 840 | Gilead | HIV infection, AIDS, ARC |
| | | (reverse transcriptase inhibitor) |
| HBY097 | Hoechst Marion | HIV infection, AIDS, ARC |
| | Roussel | (non-nucleoside reverse |
| | | transcriptase inhibitor) |
| Hypericin | VIMRx Pharm. | HIV infection, AIDS, ARC |
| Recombinant Human | Triton Biosciences | AIDS, Kaposi's sarcoma, ARC |
| Interferon Beta | (Almeda, CA) | |
| Interferon alfa-n3 | Interferon Sciences | ARC, AIDS |
| Indinavir | Merck | HIV infection, AIDS, ARC, |
| | | asymptomatic HIV positive, |
| | | also in combination with |
| | | AZT/ddI/ddC |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, AIDS, ARC |
| | | (reverse transcriptase inhibitor); |
| | | also with AZT |
| Lobucavir | Bristol-Myers Squibb | CMV infection |
| Nelfinavir | Agouron | HIV infection, AIDS, ARC |
| | Pharmaceuticals | (protease inhibitor) |
| Nevirapine | Boeheringer | HIV infection, AIDS, ARC |
| | Ingleheim | (RT inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV |
| Phosphonoformate | Products, Inc. | infection, other CMV |
| | | infections |
| PNU-140690 | Pharmacia Upjohn | HIV infection, AIDS, ARC |
| | | (protease inhibitor) |
| Probucol | Vyrex | HIV infection, AIDS |
| RBC-CD4 | Sheffield Med. | HIV infection, AIDS, ARC |
| | Tech (Houston, TX) | |
| Ritonavir | Abbott | HIV infection, AIDS, ARC |
| | | (protease inhibitor) |
| Saquinavir | Hoffmann-LaRoche | HIV infection, AIDS, ARC |
| | | (protease inhibitor) |
| Stavudine; d4T | Bristol-Myers Squibb | HIV infection, AIDS, ARC |
| Didehydrodeoxy- | | |
| Thymidine | | |
| Tipranavir | Boehringer Ingelheim | HIV infection, AIDS, ARC |
| | | (protease inhibitor) |
| Valaciclovir | Glaxo Wellcome | Genital HSV & CMV |
| | | Infections |
| Virazole | Viratek/ICN | asymptomatic HIV |
| Ribavirin | (Costa Mesa, CA) | positive, LAS, ARC |
| VX-478 | Vertex | HIV infection, AIDS, ARC |
| Zalcitabine | Hoffmann-LaRoche | HIV infection, AIDS, |
| | | ARC, with AZT |
| Zidovudine; AZT | Glaxo Wellcome | HIV infection, AIDS, ARC, |
| | | Kaposi's sarcoma, in combination |
| | | with other therapies |
| Tenofovir disoproxil, | Gilead | HIV infection, AIDS, |
| fumarate salt (VIREAD ®) | | (reverse transcriptase inhibitor) |
| EMTRIVA ® (Emtricitabine) | Gilead | HIV infection, AIDS, |
| (FTC) | | (reverse transcriptase inhibitor) |
| COMBIVIR ® | GSK | HIV infection, AIDS, |
| | | (reverse transcriptase inhibitor) |
| Abacavir succinate | GSK | HIV infection, AIDS, |
| (or ZIAGEN ®) | | (reverse transcriptase inhibitor) |
| REYATAZ ® | Bristol-Myers Squibb | HIV infection AIDs, |
| (or atazanavir) | | protease inhibitor |
| FUZEON ® | Roche/Trimeris | HIV infection AIDs, |
| (Enfuvirtide or T-20) | | viral Fusion inhibitor |
| LEXIVA ® | GSK/Vertex | HIV infection AIDs, |
| (or Fosamprenavir calcium) | | viral protease inhibitor |
| SELZENTRY ™ | Pfizer | HIV infection AIDs, (CCR5 |
| Maraviroc; (UK 427857) | | antagonist, in development) |
| TRIZIVIR ® | GSK | HIV infection AIDs, |
| | | (three drug combination) |
| Sch-417690 (vicriviroc) | Schering-Plough | HIV infection AIDs, (CCR5 |
| | | antagonist, in development) |
| TAK-652 | Takeda | HIV infection AIDs, (CCR5 |
| | | antagonist, in development) |
| GSK 873140 | GSK/ONO | HIV infection AIDs, (CCR5 |
| (ONO-4128) | | antagonist, in development) |
| Integrase Inhibitor | Merck | HIV infection AIDs |
| MK-0518 | | |
| Raltegravir | | |
| TRUVADA ® | Gilead | Combination of Tenofovir |
| | | disoproxil fumarate salt |
| | | (VIREAD ®) and EMTRIVA ® |
| | | (Emtricitabine) |
| Integrase Inhibitor | Gilead/Japan Tobacco | HIV Infection AIDs |
| GS917/JTK-303 | | in development |
| Elvitegravir | | |
| Triple drug combination | Gilead/Bristol-Myers Squibb | Combination of Tenofovir |
| ATRIPLA ® | | disoproxil fumarate salt |
| | | (VIREAD ®), EMTRIVA ® |
| | | (Emtricitabine), and |
| | | SUSTIVA ® (Efavirenz) |
| FESTINAVIR ® | Oncolys BioPharma | HIV infection AIDs |
| | | in development |
| CMX-157 | Chimerix | HIV infection AIDs |
| Lipid conjugate of | | |
| nucleotide tenofovir | | |
| GSK1349572 | GSK | HIV infection AIDS |
| Integrase inhibitor | | |
| TIVICAY ® | | |
| dolutegravir | | |

IMMUNOMODULATORS

| | | |
|---|---|---|
| AS-101 | Wyeth-Ayerst | AIDS |
| Bropirimine | Pharmacia Upjohn | Advanced AIDS |
| Acemannan | Carrington Labs, Inc. | AIDS, ARC |
| | (Irving, TX) | |
| CL246,738 | Wyeth | AIDS, Kaposi's sarcoma |
| | Lederle Labs | |
| FP-21399 | Fuki ImmunoPharm | Blocks HIV fusion |
| | | with CD4+ cells |
| Gamma Interferon | Genentech | ARC, in combination w/TNF |
| | | (tumor necrosis factor) |
| Granulocyte | Genetics Institute | AIDS |
| Macrophage Colony | Sandoz | |
| Stimulating Factor | | |
| Granulocyte | Hoechst-Roussel | AIDS |
| Macrophage Colony | Immunex | |
| Stimulating Factor | | |
| Granulocyte | Schering-Plough | AIDS, combination |
| Macrophage Colony | | w/AZT |
| Stimulating Factor | | |
| HIV Core Particle | Rorer | Seropositive HIV |
| Immunostimulant | | |
| IL-2 | Cetus | AIDS, in combination w/AZT |
| Interleukin-2 | | |
| IL-2 | Hoffman-LaRoche | AIDS, ARC, HIV, in |
| Interleukin-2 | Immunex | combination w/AZT |
| IL-2 | Chiron | AIDS, increase in |
| Interleukin-2 | | CD4 cell counts |
| (aldeslukin) | | |
| Immune Globulin | Cutter Biological | Pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's |
| | (New Orleans, LA) | sarcoma, ARC, PGL |
| Imuthiol Diethyl | Merieux Institute | AIDS, ARC |
| Dithio Carbamate | | |
| Alpha-2 | Schering Plough | Kaposi's sarcoma |
| Interferon | | w/AZT, AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl-Tripeptide | | |
| Granulocyte Colony | Amgen | AIDS, in combination |
| Stimulating Factor | | w/AZT |
| Remune | Immune Response | Immunotherapeutic |
| | Corp. | |
| rCD4 Recombinant | Genentech | AIDS, ARC |
| Soluble Human CD4 | | |
| rCD4-IgG hybrids | | AIDS, ARC |
| Recombinant | Biogen | AIDS, ARC |
| Soluble Human CD4 | | |
| Interferon | Hoffman-La Roche | Kaposi's sarcoma AIDS, ARC, |
| Alfa 2a | | in combination w/AZT |
| SK&F106528 | Smith Kline | HIV infection |
| Soluble T4 | | |
| Thymopentin | Immunobiology | HIV infection |
| | Research Institute | |
| | (Annandale, NJ) | |
| Tumor Necrosis | Genentech | ARC, in combination |
| Factor; TNF | | w/gamma Interferon |

ANTI-INFECTIVES

| | | |
|---|---|---|
| Clindamycin with | Pharmacia Upjohn | PCP |
| Primaquine | | |
| Fluconazole | Pfizer | Cryptococcal meningitis, |
| | | candidiasis |
| Pastille | Squibb Corp. | Prevention of |
| Nystatin Pastille | | oral candidiasis |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | Antibacterial |
| Trimethoprim/sulfa | | Antibacterial |
| Piritrexim | Burroughs Wellcome | PCP treatment |
| Pentamidine Isethionate | Fisons Corporation | PCP prophylaxis |
| for Inhalation | | |
| Spiramycin | Rhone-Poulenc | Cryptosporidial |
| | diarrhea | |
| Intraconazole- | Janssen-Pharm. | Histoplasmosis; cryptococcal |
| R51211 | | meningitis |
| Trimetrexate | Warner-Lambert | PCP |
| Daunorubicin | NeXstar, Sequus | Kaposi's sarcoma |
| Recombinant Human | Ortho Pharm. Corp. | Severe anemia assoc. |
| Erythropoietin | | with AZT therapy |
| Recombinant Human | Serono | AIDS-related |
| Growth Hormone | | wasting, cachexia |
| Megestrol Acetate | Bristol-Myers Squibb | Treatment of anorexia |
| | | assoc. W/AIDS |
| Testosterone | Alza, Smith Kline | AIDS-related wasting |
| Total Enteral | Norwich Eaton | Diarrhea and malabsorption |
| Nutrition | Pharmaceuticals | related to AIDS |

### Methods of Synthesis

The compounds of this invention can be made by various methods known in the art including those of the following schemes and in the specific embodiments section. The structure numbering and variable numbering shown in the synthetic schemes are distinct from, and should not be confused with, the structure or variable numbering in the claims or the rest of the specification. The variables in the schemes are meant only to illustrate how to make some of the compounds of this invention. The disclosure is not limited to the foregoing illustrative examples and the examples should be considered in all respects as illustrative and not restrictive, reference being made to the appended claims, rather than to the foregoing examples, and all changes which come within the meaning and range of equivalency of the claims are therefore intended to be embraced.

Abbreviations used in the schemes and examples generally follow conventions used in the art. Chemical abbreviations used in the specification and examples are defined as follows: “KHMDS” for potasium bis(trimethylsilyl)amide; “DMF” for N,N-dimethylformamide; “HATU” for O-(t-Azabenzotriazol-1-yl)-N,N,N′,N′-tetramethyluronium hexafluorophosphate, “MeOH” for methanol; “Ar” for aryl; “TFA” for trifluoroacetic acid, “DMSO” for dimethylsulfoxide; “h” for hours; “rt” for room temperature or retention time (context will dictate); “min” for minutes; “EtOAc” for ethyl acetate; “THF” for tetrahydrofuran; “Et₂O” for diethyl ether; “DMAP” for 4-dimethylaminopyridine; “DCE” for 1,2-dichloroethane; “ACN” for acetonitrile; “DME” for 1,2-dimethoxyethane; “HOBt” for 1-hydroxybenzotriazole hydrate; and “DIEA” for diisopropylethylamine.

Certain other abbreviations as used herein, are defined as follows: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “atm” for atmosphere, “psi” for pounds per square inch, “conc.” for concentrate, “sat” or “sat'd ” for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry , “LCMS” for liquid chromatography mass spectrometry, “HPLC” for high pressure liquid chromatography, “RP HPLC” for reverse phase HPLC, “TLC” or “tic” for thin layer chromatography, “NMR” for nuclear magnetic resonance spectroscopy, “¹H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.

Some compounds can be synthesized from an appropriately substituted heterocycle I-1 according to Scheme I, Compounds I-1 and I-6 are commercially available or synthesized by reactions well known in the art. Treatment of compound I-1 with bromine provided the dibromo intermediates I-2 which was converted to the chloropyridine I-3 by reacting with POCl₃. Intermediate I-3 conveniently transformed to ketoester I-5 using conditions well-known to those skilled in the art, including reacting I-3 with Grignard reagent in the presence of catalytic copper(I) bromide dimethylsulfide complex followed by alkyl 2-chloro-2-oxoacetate. Coupling of amines I-5 with intermediate I-6 in the presence of an organic base such as Hunig's base provided intermediate I-7. Chiral Lewis acid such as I-8 mediated reduction of ketoester I-7 with catecholborane furnished chiral alcohol I-9. Tertiary butylation of alcohol I-9 by well-known conditions, including but not limited to isobutylene and perchloric acid, gave intermediate I-10. Intermediate I-10 are conveniently transformed to intermediate I-11 using conditions well-known in the art, including but not limited to the Suzuki coupling between intermediate I-10 and R⁶B(OR)₂. The boronate or boronic acid coupling reagents, well-known in the art, are commercially available or are prepared by reactions well-known to those skilled in the art. Hydrolysis of intermediate I-11 by using conditions well-known to those skilled in the art furnished carboxylic acid I-12.

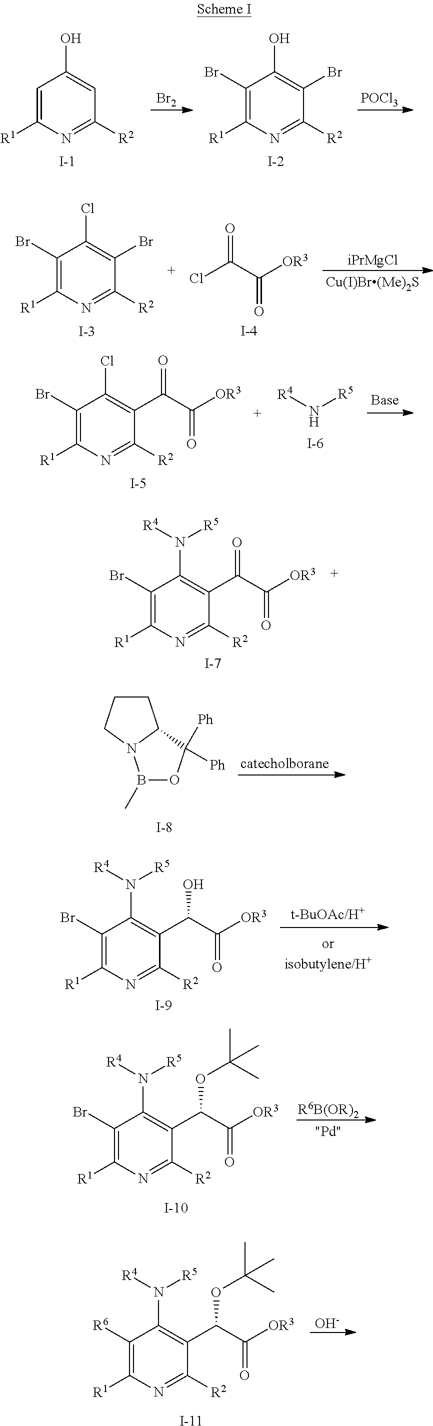

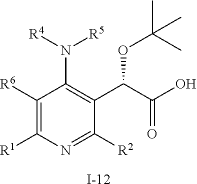

Intermediates I-10 are conveniently transformed to intermediates II-2 using conditions well-known in the art, including but not limited to the Suzuki coupling between intermediates I-10 and II-1. Cleavage of protecting group in II-2 provided amine II-3. Alkylation of the amine II-3 was achieved by using conditions well known to those skilled in the art, including but not limited to reductive alkylation to provide the intermediate II-4. Hydrolysis of intermediate II-4 by using conditions well-known in the literature furnished carboxylic acid II-5.

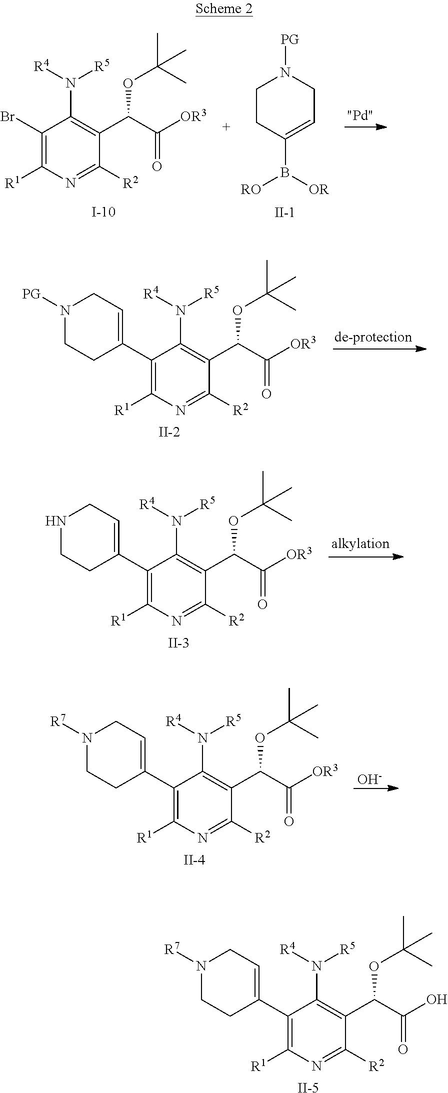

The compounds described herein were purified by the methods well known to those skilled in art by normal phase column chromatography on silica gel column using appropriate solvent system described. Preparative HPLC purifications mentioned in this experimentation section were carried out gradient elution either on Sunfire Prep C18 ODB column (5 μm; 19 or 30×100 mm) or Waters Xbridge column (5 μM; 19 or 30×100 mm) using the following mobile phases: Mobile phase A: 9:1 H₂O/acetonitrile with 10 mM NH₄OAc and mobile phase B: A: 9:1 acetonitrile/H₂O with: 10 mM NH₄OAc; or mobile phase A: 9:1 H₂O/acetonitrile with 0.1% TFA and mobile phase B: A: 9:1 acetonitrile/H₂O with: 0.1% TFA; or mobile phase A: water with 20 mM NH₄OAc and mobile phase B: 95:5 MeOH/H₂O with 20 mM NH₄OAc.

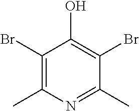

3,5-Dibromo-2,6-dimethylpyridin-4-ol: A 3-neck R.B-flask equipped with mechanical stirrer, addition funnel and condenser is charged with 2,6-dimethylpyridin-4-ol (100 g, 812 mmol), CH₂Cl₂ (1000 mL) and MeOH (120 mL). To the resulting light brown or tan solution was added tert-BuNH2 (176 ml, 1665 mmol), cooled in water bath maintained between 5-10° C. (ice-water) and added drop wise Br2 (84 ml, 1624 mmol) over 70 min. After the addition was complete cold bath was removed and stirred for 1.5 h at rt. Then, the light orange slurry was filtered and the filter cake was washed with ether (250 mL) and dried to afford 3,5-dibromo-2,6-dimethylpyridin-4-ol, hydrobromide (280.75 g, 776 mmol, 96% yield) as white solid which was used in the next step without further purification. ¹H NMR (500 MHz, DMSO-d₆) δ 12.08 (br. s., 1H), 2.41 (s, 6H). LCMS (M+H)=281.9.

Alternative procedure: Bromine (72.8 mL, 1.4 mol) was added via addition funnel over 60 min to a mechanically stirred cold (ice-water bath) solution of 2,6-dimethylpyridin-4-ol (87 g, 706 mmol) and 4-methylmorpholine (156 mL, 1.4 mol) in dichloromethane (1 L) and methanol (100 mL) and then stirred for 2 h at rt. Additional bromine (˜15 mL) was added based on monitoring by LCMS. The product was filtered, washed with ether, and dried under vacuum to give 3,5-dibromo-2,6-dimethylpyridin-4-ol 176.8 g (88%).

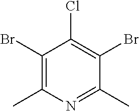

3,5-Dibromo-4-chloro-2,6-dimethyl-pyridine: Triethylamine (28.8 mL, 206 mmol) was added to a nitrogen purged solution of 3,5-dibromo-2,6-dimethylpyridin-4-ol (58 g, 206 mmol) and phosphorous oxychloride (57.7 mL, 619 mmol) in chloroform (450 mL) and stirred for 1 h at rt, then 3 h at 80° C. The reaction was removed from heating and immediately concentrated under house vaccum; then under high vacuum. The appearance was a cream colored solid, which was azeotroped with toluene (2×100 mL); treated with ice (200 g) for 10 min and carefully neutralized with NaHCO₃ (powder), and 1N NaOH solution, and extracted with DCM (2×400 mL). The combined organic layers were dried (MgSO₄), concentrated, and a beige solid was obtained that was washed with hexanes and dried under high vacuum to give 3,5-dibromo-4-chloro-2,6-dimethyl-pyridine 52.74 g (85.1%). Concentration of the hexanes gave 3.5 g of less pure product. ¹H NMR (500 MHz, CDCl₃) δ 2.59 (s, 6H). LCMS (M+H)=300.0.

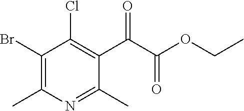 [SMILES: CCOC(=O)C(=O)c1c(C)nc(C)c(Br)c1Cl]

Ethyl 2-(5-bromo-4-chloro-2,6-dimethylpyridin-3-yl)-2-oxoacetate: To a stirred mixture of 3,5-dibromo-4-chloro-2,6-dimethylpyridine (14.94 g, 49.9 mmol) and Cu(I)Br Me2S (0.513 g, 2.495 mmol) in THF (50 mL) was added drop wise 2M iPrMgCl/THF (26.2 ml, 52.4 mmol) at −30° C. over 5 min. Then, the resulting slurry was warmed to −10° C. over 30 min and stirred for 30 min. The homogeneous brown reaction mixture was rapidly transferred via cannula to a solution of ethyl 2-chloro-2-oxoacetate (6.14 ml, 54.9 mmol, degassed for 5 min by bubbling N2 through the solution) in THF (50 mL) maintained at −30° C. The resulting reaction mixture was stirred (1.5 h) while warming to 0° C. Then, taken up in to Et₂O (200 mL), washed with 1:1 sat Na₂CO₃/1M NH₄Cl (3×50 mL), dried (MgSO₄), filtered and concentrated to give brown viscous oil. Flash chromatography using 2.5, 5 and 7.5% EtOAc/Hex afforded ethyl 2-(5-bromo-4-chloro-2,6-dimethylpyridin-3-yl)-2-oxoacetate (14.37 g, 44.8 mmol, 90% yield) as white solid. ¹H NMR (400 MHz, CDCl₃) δ 4.42 (q, J=7.0 Hz, 2H), 2.76 (s, 3H), 2.46 (s, 3H), 1.41 (t, J=7.2 Hz, 3H). LCMS (M+H)=322.1.

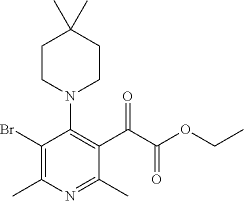 [SMILES: CCOC(=O)C(=O)c1c(C)nc(C)c(Br)c1N1CCC(C)(C)CC1]

Ethyl 2-(5-bromo-4-(4,4-dimethylpiperidin-1-yl)-2,6-dimethylpyridin-3-yl)-2-oxoacetate: To a solution of 4,4-dimethylpiperidine (1.245 g, 11.00 mmol) and DIEA (3.49 ml, 20.00 mmol) in anhydrous CH₃CN (40 mL) was added ethyl 2-(5-bromo-4-chloro-2,6-dimethylpyridin-3-yl)-2-oxoacetate (3.21 g, 10 mmol) at rt. The resulting mixture was placed in a pre-heated oil bath (80° C.). After 22 h, the reaction mixture was concentrated and the residue was purified by flash chromatography using 1-lit each 2.5, 5, 7.5 and 10% EtOAc/Hex to afford ethyl 2-(5-bromo-4-(4,4-dimethylpiperidin-1-yl)-2,6-dimethylpyridin-3-yl)-2-oxoacetate (2.846 g, 7.16 mmol, 71.6% yield) as yellow solid. ¹H NMR (500 MHz, CDCl₃) δ 4.37 (q, J=7.1 Hz, 2H), 3.67-2.75 (br.s., 4H), 2.71 (s, 3H), 2.44 (s, 3H), 1.42 (t, J=7.1 Hz, 3H), 1.38 (t, J=5.6 Hz, 4H), 1.00 (s, 6H). LCMS (M+H)=399.4.

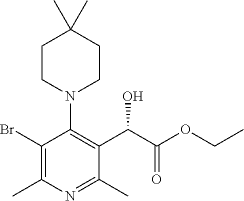 [SMILES: CCOC(=O)[C@@H](O)c1c(C)nc(C)c(Br)c1N1CCC(C)(C)CC1]

(S)-Ethyl 2-(5-bromo-4-chloro-2,6-dimethylpyridin-3-yl)-2-hydroxyacetate: To stirred yellow solution of ethyl 2-(5-bromo-4-(4,4-dimethylpiperidin-1-yl)-2,6-dimethylpyridin-3-yl)-2-oxoacetate (2.25 g, 5.66 mmol) and (R)-1-methyl-3,3-diphenylhexahydropyrrolo[1,2-c][1,3,2]oxazaborole (0.314 g, 1.133 mmol) in toluene (30 mL) at −35° C. was added drop wise 50% catecholborane (1.819 ml, 8.49 mmol) over 10 min. The reaction mixture was slowly warmed to −15° C. over 1 h and then left for 2 h at −15° C. Then, diluted with EtOAc (100 mL), washed with sat Na₂CO₃ (4×25 mL) by vigorously stirring and separating aqueous layers. The organic layer dried (MgSO₄), filtered, concentrated and purified by flash chromatography using 10, 20 and 25% EtOAc/Hex to afford desired (S)-ethyl 2-(5-bromo-4-(4,4-dimethylpiperidin-1-yl)-2,6-dimethylpyridin-3-yl)-2-hydroxyacetate (2.2596 g, 5.66 mmol, 100% yield) contaminated with about 10% of (S)-ethyl 2-(5-bromo-4-chloro-2,6-dimethylpyridin-3-yl)-2-hydroxyacetate. Used in the next step without further purification. ¹H NMR (500 MHz, CDCl₃) δ 5.71 (d, J=7.3 Hz, 1H), 5.54 (d, J=7.4 Hz, 1H), 4.29 (dq, J=10.8, 7.1 Hz, 1H), 4.16 (dq, J=10.8, 7.1 Hz, 1H), 3.94-3.83 (m, 2H), 2.71 (d, J=11.9 Hz, 1H), 2.67 (s, 3H), 2.59 (s, 3H), 2.54 (d, J=12.0 Hz, 1H), 1.71 (td, J=12.7, 4.7 Hz, 1H), 1.62 (td, J=13.0, 4.7 Hz, 1H), 1.42 (dd, J=13.1, 2.2 Hz, 1H), 1.37 (dd, J=12.9, 2.4 Hz, 1H), 1.25 (t, J=7.1 Hz, 3H), 1.09 (s, 3H), 1.04 (s, 3H). LCMS (M+H)=401.3.

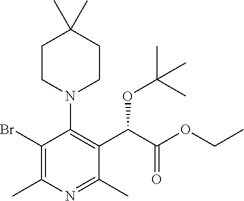 [SMILES: CCOC(=O)[C@@H](OC(C)(C)C)c1c(C)nc(C)c(Br)c1N1CCC(C)(C)CC1]

(S)-Ethyl 2-(5-bromo-4-(4,4-dimethylpiperidin-1-yl)-2,6-dimethylpyridin-3-yl)-2-(tert-butoxy)acetate: A stirred ice-cold yellow mixture of (S)-ethyl 2-(5-bromo-4-(4,4-dimethylpiperidin-1-yl)-2,6-dimethylpyridin-3-yl)-2-hydroxyacetate (2.45 g, 6.14 mmol) and 70% HClO₄ (1.054 ml, 12.27 mmol) in CH₂Cl₂ (100 mL) was saturated with isobutylene gas by bubbling through the reaction mixture (10 min). After 2 h, cold bath was removed and the turbid reaction mixture stirred for 22 h at rt. LCMS at this point showed 4:1 product to sm. So, saturated with isobutylene (5 min) at rt and stirred for additional 24 h. Then, neutralized with sat. Na₂CO₃ (30 mL), organic layer separated and aqueous layer extracted with CH₂Cl₂ (25 mL). The combined organic layers dried (MgSO₄), filtered, concentrated and purified by flash chromatography using 5, 10, 15, 20 and 40% EtOAc/hex to afford (S)-ethyl 2-(5-bromo-4-(4,4-dimethylpiperidin-1-yl)-2,6-dimethylpyridin-3-yl)-2-(tert-butoxy)acetate (2.3074 g, 5.07 mmol, 83% yield) as yellow oil: ¹H NMR (500 MHz, CDCl₃) δ 6.19 (br. s., 1H), 4.17-4.24 (m, 1H), 4.08-4.14 (m, 1H), 4.04 (dt, J=2.5, 12.1 Hz, 1H), 3.51 (dt, J=2.5, 12.1 Hz, 1H), 2.85-2.91 (m, 1H), 2.64 (s, 3H), 2.57-2.62 (m, 1H), 2.55 (s, 3H), 1.55-1.66 (m, 2H), 1.41-1.46 (m, 1H), 1.32-1.37 (m, 1H), 1.21 (s, 9H), 1.20 (t, J=7.2 Hz, 2H), 1.08 (s, 3H), 1.03 (s, 3H). LCMS (M+H)=457.4. And (S)-ethyl 2-(5-bromo-4-(4,4-dimethylpiperidin-1-yl)-2,6-dimethylpyridin-3-yl)-2-hydroxyacetate (0.3 g, 0.751 mmol, 12.24% yield) as pale yellow paste: LCMS (M+H)=401.3.

### EXAMPLE 1

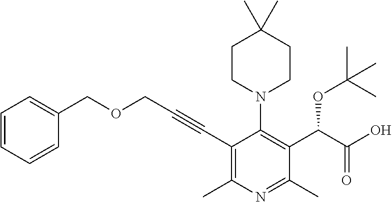 [SMILES: Cc1nc(C)c([C@H](OC(C)(C)C)C(=O)O)c(N2CCC(C)(C)CC2)c1C#CCOCc1ccccc1]

(S)-2-(5-(3-(Benzyloxy)prop-1-yn-1-yl)-4-(4,4-dimethylpiperidin-1-yl)-2,6-dimethylpyridin-3-yl)-2-(tert-butoxy)acetic acid: A mixture of (S)-ethyl 2-(5-bromo-4-(4,4-dimethylpiperidin-1-yl)-2,6-dimethylpyridin-3-yl)-2-(tert-butoxy)acetate (0.0615 g, 0.135 mmol), ((prop-2-yn-1-yloxy)methyl)benzene (0.039 g, 0.270 mmol), TBAF.3H₂O (0.3152 g, 1.206 mmol) and (Ph₃P)₂PdCl₂ (9.48 mg, 0.014 mmol) was heated under N2 atm for 18 h at 75-80° C. Then, diluted with ether (25 mL), washed with water (2×5 mL), brine (5 mL), dried (MgSO₄), filtered and concentrated to give crude product. LCMS analysis showed presence of both ester and carboxylic acid. A mixture of above crude residue and LiOH (0.032 g, 1.350 mmol) in 9:1 EtOH/H₂O was refluxed for 4 h. Then, cooled and purified by prep-HPLC to afford (S)-2-(5-(3-(benzyloxy)prop-1-yn-1-yl)-4-(4,4-dimethylpiperidin-1-yl)-2,6-dimethylpyridin-3-yl)-2-(tert-butoxy)acetic acid (0.0063 g, 0.013 mmol, 9.47% yield) as colorless paste. ¹H NMR (500 MHz, CDCl₃) δ 7.38-7.41 (m, 4H), 7.32-7.36 (m, 1H), 5.48-6.01 (br.s., 1H), 4.71 (s, 2H), 4.52 (s, 2H), 2.68 (s, 3H), 2.65 (s, 3H), 1.41-1.61 (m, 4H), 1.27 (s, 9H), 1.04 (s, 6H). 4H of piperidine are missing. LCMS (M+H)=493.5.

### EXAMPLE 2

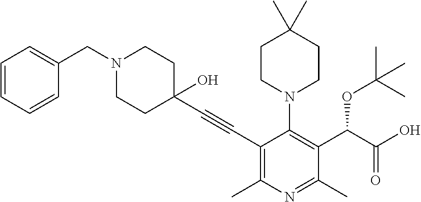 [SMILES: Cc1nc(C)c([C@H](OC(C)(C)C)C(=O)O)c(N2CCC(C)(C)CC2)c1C#CC1(O)CCN(Cc2ccccc2)CC1]

(S)-2-(5-((1-Benzyl-4-hydroxypiperidin-4-yl)ethynyl)-4-(4,4-dimethylpiperidin-1-yl)-2,6-dimethylpyridin-3-yl)-2-(tert-butoxy)acetic acid: A mixture of (S)-ethyl 2-(5-bromo-4-(4,4-dimethylpiperidin-1-yl)-2,6-dimethylpyridin-3-yl)-2-(tert-butoxy)acetate (0.0629 g, 0.138 mmol),1-benzyl-4-ethynylpiperidin-4-ol (0.030 g, 0.138 mmol), TBAF.3H₂O (0.348 g, 1.105 mmol) and (Ph₃P)₂PdCl₂ (9.69 mg, 0.014 mmol) was heated under N2 for 18 h at 80-85° C. LCMS at this point showed presnce of carboxylic acid of the desired compound. Then, diluted with EtOAc (25 mL), washed with water (2×5 mL), brine (5 mL), dried (MgSO₄), filtered and concentrated to give brown residue which was purified by prep-HPLC to afford (S)-2-(5-((1-benzyl-4-hydroxypiperidin-4-yl)ethynyl)-4-(4,4-dimethylpiperidin-1-yl)-2,6-dimethylpyridin-3-yl)-2-(tert-butoxy)acetic acid as solid. LCMS (M+H)=562.6.

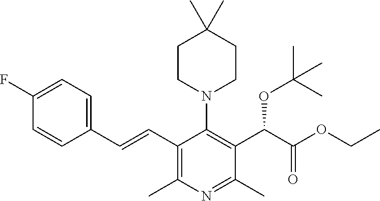 [SMILES: CCOC(=O)[C@@H](OC(C)(C)C)c1c(C)nc(C)c(/C=C/c2ccc(F)cc2)c1N1CCC(C)(C)CC1]

(S,E)-Ethyl 2-(tert-butoxy)-2-(4-(4,4-dimethylpiperidin-1-yl)-5-(4-fluorostyryl)-2,6-dimethylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.0555 g, 0.122 mmol), (E)-(4-fluorostyryl)boronic acid (0.030 g, 0.183 mmol) and 2M Na₂CO₃ (0.152 ml, 0.305 mmol) in DMF (3 mL) was degassed for 10 min. Then, Pd(Ph₃P)₄ (0.014 g, 0.012 mmol) was added, degassed for 5 min and placed in a pre-heated oil bath at 100° C. After 2 h at 110° C., the reaction mixture was cooled and purified by prep-HPLC to afford (S,E)-ethyl 2-(tert-butoxy)-2-(4-(4,4-dimethylpiperidin-1-yl)-5-(4-fluorostyryl)-2,6-dimethylpyridin-3-yl)acetate (0.0438 g, 0.088 mmol, 72.4% yield) as off-white solid/foam. ¹H NMR (500 MHz, CDCl₃) δ 7.47-7.53 (m, 2H), 7.05-7.15 (m, 3H), 6.48 (d, J=16.6 Hz, 1H), 6.07 (br. S., 1H), 4.13-4.30 (m, 2H), 2.61 (s, 3H), 2.53 (s, 3H), 1.58-1.71 (m, 4H), 1.25 (t, J=7.1 Hz, 3H), 1.21 (s, 9H), 0.94 (br.s., 6H). 4H of piperidine are missing. LCMS (M+H)=497.6.

### EXAMPLE 3

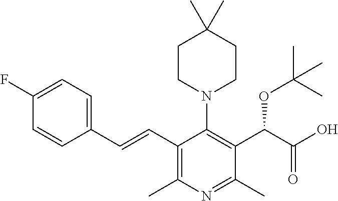 [SMILES: Cc1nc(C)c([C@H](OC(C)(C)C)C(=O)O)c(N2CCC(C)(C)CC2)c1/C=C/c1ccc(F)cc1]

(S,E)-2-(tert-Butoxy)-2-(4-(4,4-dimethylpiperidin-1-yl)-5-(4-fluorostyryl)-2,6-dimethylpyridin-3-yl)acetic acid: A mixture of (S,E)-ethyl 2-(tert-butoxy)-2-(4-(4,4-dimethylpiperidin-1-yl)-5-(4-fluorostyryl)-2,6-dimethylpyridin-3-yl)acetate (0.04 g, 0.081 mmol) and LiOH (0.019 g, 0.805 mmol) in 9:1 EtOH/H₂O (2 mL) was refluxed for 2.5 h. Then, cooled and purified by prep-HPLC to afford (S,E)-2-(tert-butoxy)-2-(4-(4,4-dimethylpiperidin-1-yl)-5-(4-fluorostyryl)-2,6-dimethylpyridin-3-yl)acetic acid (0.0336 g, 0.072 mmol, 89% yield) as white solid. ¹H NMR (500 MHz, CDCl₃) δ 7.45-7.51 (m, 2H), 7.07-7.13 (m, 2H), 6.97 (d, J=16.6 Hz, 1H), 6.46 (d, J=16.6 Hz, 1H), 5.79 (br. s., 1H), 3.58-3.69 (m, 2H), 2.86 (m, 2H), 2.81-2.93 (s, 3H), 2.56 (s, 3H), 1.41-1.58 (m, 4H), 1.24 (s, 9H), 0.92 (s, 6H). LCMS (M+H)=469.5.

### EXAMPLE 4

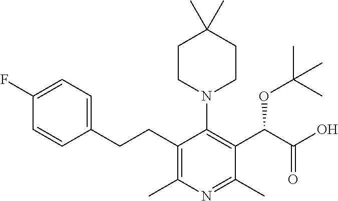 [SMILES: Cc1nc(C)c([C@H](OC(C)(C)C)C(=O)O)c(N2CCC(C)(C)CC2)c1CCc1ccc(F)cc1]

(S)-2-(tert-Butoxy)-2-(4-(4,4-dimethylpiperidin-1-yl)-5-(4-fluorophenethyl)-2,6-dimethylpyridin-3-yl)acetic acid: A mixture of (S,E)-2-(tert-butoxy)-2-(4-(4,4-dimethylpiperidin-1-yl)-5-(4-fluorostyryl)-2,6-dimethylpyridin-3-yl)acetic acid (0.009 g, 0.019 mmol) and 10% Pd/C (2.044 mg, 1.921 μmol) in 1:1 MeOH/EtOAc (5 mL) was left under balloon H₂ atmoshpere for 1 h. Then, filtered through a plug of celite and concentrated to give (S)-2-(tert-butoxy)-2-(4-(4,4-dimethylpiperidin-1-yl)-5-(4-fluorophenethyl)-2,6-dimethylpyridin-3-yl)acetic acid (0.009 g, 0.019 mmol, 99% yield) as colorless paste. ¹H NMR (500 MHz, CDCl₃) δ 7.19-7.23 (m, 2H), 7.01-7.07 (m, 2H), 5.89 (br. s., 1H), 3.32-3.60 (m, 2H), 2.74-3.25 (m, 6H), 2.65 (s, 3H), 2.63 (s, 3H), 1.33-1.79 (m, 4H), 1.26 (s, 9H), 1.05 (s, 6H). LCMS (M+H)=471.5.

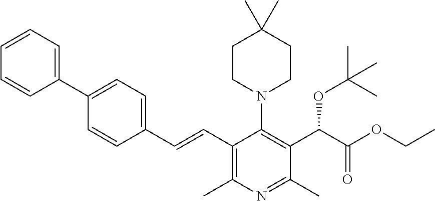 [SMILES: CCOC(=O)[C@@H](OC(C)(C)C)c1c(C)nc(C)c(/C=C/c2ccc(-c3ccccc3)cc2)c1N1CCC(C)(C)CC1]

(S,E)-Ethyl 2-(5-(2-([1,1′-biphenyl]-4-yl)vinyl)-4-(4,4-dimethylpiperidin-1-yl)-2,6-dimethylpyridin-3-yl)-2-(tert-butoxy)acetate: A mixture of (S)-ethyl 2-(5-bromo-4-(4,4-dimethylpiperidin-1-yl)-2,6-dimethylpyridin-3-yl)-2-(tert-butoxy)acetate (0.0494 g, 0.108 mmol), (E)-(2-([1,1′-biphenyl]-4-yl)vinyl)boronic acid (0.036 g, 0.163 mmol) and 2M Na₂CO₃ (0.136 ml, 0.271 mmol) in DMF (3 mL) was degassed for 10 min. Then, Pd(Ph₃P)₄ (0.013 g, 10.85 μmol) was added, degassed for 5 min and placed in a pre-heated oil bath at 100° C. After 2 h at 110° C., the reaction mixture was cooled and purified by prep-HPLC to afford (S,E)-ethyl 2-(5-(2-([1,1′-biphenyl]-4-yl)vinyl)-4-(4,4-dimethylpiperidin-1-yl)-2,6-dimethylpyridin-3-yl)-2-(tert-butoxy)acetate (0.032 g, 0.058 mmol, 53.2% yield) as off-white solid. ¹H NMR (500 MHz, CDCl₃) δ 7.65-7.69 (m, 4H), 7.60-7.63 (m, 2H), 7.47-7.51 (m, 2H), 7.37-7.41 (m, 1H), 7.22 (d, J=16.6 Hz, 1H), 6.57 (d, J=16.6 Hz, 1H), 6.08 (br. s., 1H), 4.13-4.30 (m, 2H), 2.98-3.66 (m, 4H), 2.62 (s, 3H), 2.57 (s, 3H), 1.55-1.70 (m, 4H), 1.26 (t, J=7.1 Hz, 3H), 1.22 (s, 9H), 0.96 (br. s., 6H). LCMS (M+H)=555.6.

### EXAMPLE 5

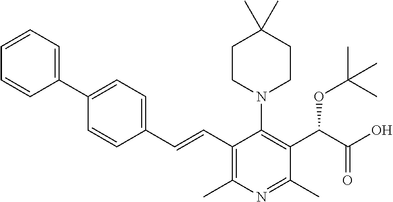 [SMILES: Cc1nc(C)c([C@H](OC(C)(C)C)C(=O)O)c(N2CCC(C)(C)CC2)c1/C=C/c1ccc(-c2ccccc2)cc1]

(S,E)-2-(5-(2-([1,1′-Biphenyl]-4-yl)vinyl)-4-(4,4-dimethylpiperidin-1-yl)-2,6-dimethylpyridin-3-yl)-2-(tert-butoxy)acetic acid: A mixture of (S,E)-ethyl 2-(5-(2-([1,1′-biphenyl]-4-yl)vinyl)-4-(4,4-dimethylpiperidin-1-yl)-2,6-dimethylpyridin-3-yl)-2-(tert-butoxy)acetate (0.029 g, 0.052 mmol) and LiOH (0.013 g, 0.523 mmol) in 9:1 EtOH/H₂O (2 mL) was refluxed for 2.5 h. Then, cooled and purified by prep-HPLC to afford (S,E)-2-(5-(2-([1,1′-biphenyl]-4-yl)vinyl)-4-(4,4-dimethylpiperidin-1-yl)-2,6-dimethylpyridin-3-yl)-2-(tert-butoxy)acetic acid (0.024 g, 0.046 mmol, 87% yield) as white solid. ¹H NMR (500 MHz, CDCl₃) δ 7.63-7.68 (m, 4H), 7.58-7.61 (m, 2H), 7.45-7.51 (m, 2H), 7.37-7.42 (m, 1H), 7.10 (d, J=16.6 Hz, 1H), 6.56 (d, J=16.6 Hz, 1H), 5.80 (br. S., 1H), 3.65-3.74 (m, 2H), 2.81-3.03 (m, 2H), 2.75 (s, 3H), 2.60 (s, 3H), 1.41-1.61 (m, 4H), 1.25 (s, 9H), 0.95 (s, 6H). LCMS (M+H)=527.5.

### EXAMPLE 6

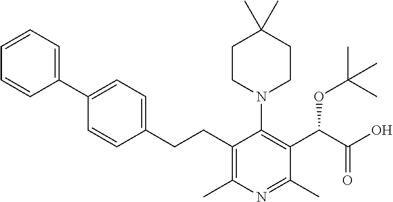 [SMILES: Cc1nc(C)c([C@H](OC(C)(C)C)C(=O)O)c(N2CCC(C)(C)CC2)c1CCc1ccc(-c2ccccc2)cc1]

(S)-2-(5-(2-([1,1′-Biphenyl]-4-yl)ethyl)-4-(4,4-dimethylpiperidin-1-yl)-2,6-dimethylpyridin-3-yl)-2-(tert-butoxy)acetic acid: A mixture of (S,E)-2-(5-(2-([1,1′-biphenyl]-4-yl)vinyl)-4-(4,4-dimethylpiperidin-1-yl)-2,6-dimethylpyridin-3-yl)-2-(tert-butoxy)acetic acid (0.0138 g, 0.026 mmol) and 10% Pd/C (2.79 mg, 2.62 μmol) in 1:1 MeOH/EtOAc (5 mL) was left under balloon H₂ atmosphere for 1 h. Then, filtered through a plug of celite and concentrated to give (S)-2-(5-(2-([1,1′-biphenyl]-4-yl)ethyl)-4-(4,4-dimethylpiperidin-1-yl)-2,6-dimethylpyridin-3-yl)-2-(tert-butoxy)acetic acid (0.0126 g, 0.022 mmol, 83% yield) as white solid. ¹H NMR (500 MHz, CDCl₃) δ 7.58-7.64 (m, 4H), 7.44-7.49 (m, 2H), 7.34-7.40 (m, 3H), 5.79 (s, 1H), 3.37-3.65 (m, 2H), 2.79-3.26 (m, 6H), 2.68 (s, 6H), 1.32-1.80 (m, 4H), 1.27 (s, 9H), 1.07 (s, 6H). LCMS (M+H)=529.5.

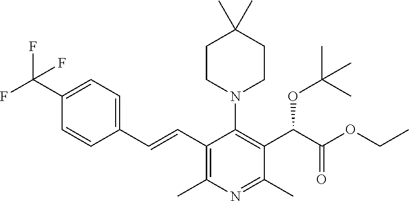 [SMILES: CCOC(=O)[C@@H](OC(C)(C)C)c1c(C)nc(C)c(/C=C/c2ccc(C(F)(F)F)cc2)c1N1CCC(C)(C)CC1]

(S,E)-Ethyl 2-(tert-butoxy)-2-(4-(4,4-dimethylpiperidin-1-yl)-2,6-dimethyl-5-(4-(trifluoromethyl)styryl)pyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.0527 g, 0.116 mmol), (E)-(4-(trifluoromethyl)styryl)boronic acid (0.037 g, 0.174 mmol) and 2M Na₂CO₃ (0.145 ml, 0.289 mmol) in DMF (3 mL) was degassed for 10 min. Then, Pd(Ph₃P)₄ (0.013 g, 0.012 mmol) added, degassed for 5 min and placed in a pre-heated oil bath at 100° C. After 2 h at 110° C., the reaction mixture was cooled and purified by prep-HPLC to afford (S,E)-ethyl 2-(tert-butoxy)-2-(4-(4,4-dimethylpiperidin-1-yl)-2,6-dimethyl-5-(4-(trifluoromethyl)styryl)pyridin-3-yl)acetate (0.048 g, 0.088 mmol, 76% yield) as white solid. ¹H NMR (500 MHz, CDCl₃) δ 7.60-7.70 (m, 4H), 7.29 (d, J=16.6 Hz, 1H), 6.57 (d, J=16.6 Hz, 1H), 6.05 (br. s., 1H), 4.13-4.30 (m, 2H), 2.91-3.52 (m, 4H), 2.61 (s, 3H), 2.53 (s, 3H), 1.52-1.78 (m, 4H), 1.26 (t, J=7.1 Hz, 3H), 1.21 (s, 9H), 0.94 (br.s., 6H). LCMS (M+H)=547.6.

### EXAMPLE 7

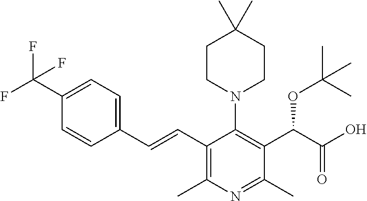 [SMILES: Cc1nc(C)c([C@H](OC(C)(C)C)C(=O)O)c(N2CCC(C)(C)CC2)c1/C=C/c1ccc(C(F)(F)F)cc1]

(S,E)-2-(tert-Butoxy)-2-(4-(4,4-dimethylpiperidin-1-yl)-2,6-dimethyl-5-(4-(trifluoromethyl)styryl)pyridin-3-yl)acetic acid: A mixture of (S,E)-ethyl 2-(tert-butoxy)-2-(4-(4,4-dimethylpiperidin-1-yl)-2,6-dimethyl-5-(4-(trifluoromethyl)styryl)pyridin-3-yl)acetate (0.04 g, 0.073 mmol) and LiOH (0.018 g, 0.732 mmol) in 9:1 EtOH/H₂O (2 mL) was refluxed for 2.5 h. Then, cooled and purified by prep-HPLC to afford (S,E)-2-(tert-butoxy)-2-(4-(4,4-dimethylpiperidin-1-yl)-2,6-dimethyl-5-(4-(trifluoromethyl)styryl)pyridin-3-yl)acetic acid (0.0352 g, 0.068 mmol, 93% yield) as white solid. ¹H NMR (500 MHz, CDCl₃) δ 7.65-7.69 (m, 2H), 7.59-7.63 (m, 2H), 7.19 (d, J=16.6 Hz, 1H), 6.55 (d, J=16.6 Hz, 1H), 5.84 (br. S., 1H), 3.55-3.66 (m, 2H), 2.80-2.96 (m, 2H), 2.70 (s, 3H), 2.56 (s, 3H), 1.40-1.57 (m, 4H), 1.24 (s, 9H), 0.93 (s, 6H). LCMS (M+H)=519.5.

### EXAMPLE 8

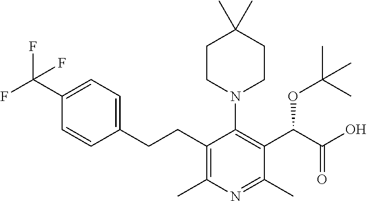 [SMILES: Cc1nc(C)c([C@H](OC(C)(C)C)C(=O)O)c(N2CCC(C)(C)CC2)c1CCc1ccc(C(F)(F)F)cc1]

(S)-2-(tert-Butoxy)-2-(4-(4,4-dimethylpiperidin-1-yl)-2,6-dimethyl-5-(4-(trifluoromethyl)phenethyl)pyridin-3-yl)acetic acid: A mixture of (S,E)-2-(tert-butoxy)-2-(4-(4,4-dimethylpiperidin-1-yl)-2,6-dimethyl-5-(4-(trifluoromethyl)styryl)pyridin-3-yl)acetic acid (0.0107 g, 0.021 mmol) and 10% Pd/C (2.196 mg, 2.063 μmol) in 1:1 MeOH/EtOAc (5 mL) was left under balloon H₂ atmosphere for 1 h. Then, filtered through a plug of celite and concentrated to give (S)-2-(tert-butoxy)-2-(4-(4,4-dimethylpiperidin-1-yl)-2,6-dimethyl-5-(4-(trifluoromethyl)phenethyl)pyridin-3-yl)acetic acid (0.0106 g, 0.020 mmol, 96% yield) as colorless paste. ¹H NMR (500 MHz, CDCl₃) δ 7.62 (d, J=8.0 Hz, 2H), 7.38 (d, J=8.0 Hz, 2H), 5.89 (br. s., 1H), 3.31-3.58 (m, 2H), 2.79-3.22 (m, 6H), 2.66 (s, 3H), 2.65 (s, 3H), 1.36-1.79 (m, 4H), 1.26 (s, 9H), 1.04 (s, 6H). LCMS (M+H)=521.5.

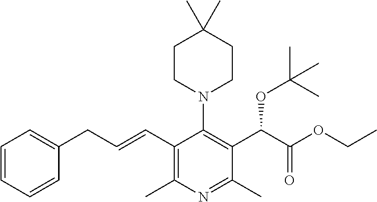 [SMILES: CCOC(=O)[C@@H](OC(C)(C)C)c1c(C)nc(C)c(/C=C/Cc2ccccc2)c1N1CCC(C)(C)CC1]

(S,E)-Ethyl 2-(tert-butoxy)-2-(4-(4,4-dimethylpiperidin-1-yl)-2,6-dimethyl-5-(3-phenylprop-1-en-1-yl)pyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.057 g, 0.125 mmol), (E)-(3-phenylprop-1-en-1-yl)boronic acid (0.030 g, 0.188 mmol) and 2M Na₂CO₃ (0.156 ml, 0.313 mmol) in DMF (3 mL) was degassed for 10 min. Then, Pd(Ph₃P)₄ (0.014 g, 0.013 mmol) added, degassed for 5 min and placed in a pre-heated oil bath at 100° C. After 2 h at 110° C., cooled and purified by prep-HPLC to afford (S,E)-ethyl 2-(tert-butoxy)-2-(4-(4,4-dimethylpiperidin-1-yl)-2,6-dimethyl-5-(3-phenylprop-1-en-1-yl)pyridin-3-yl)acetate (0.0384 g, 0.078 mmol, 62.3% yield) as pale yellow paste. ¹H NMR (500 MHz, CDCl₃) δ 7.32-7.37 (m, 2H), 7.27-7.31 (m, 2H), 7.22-7.27 (m, 1H), 6.45 (d, J=16.1 Hz, 1H), 6.06 (s, 1H), 5.78 (td, J=6.6, 16.0 Hz, 1H), 4.09-4.25 (m, 2H), 3.57-3.69 (m, 2H), 3.45 (br. s., 1H), 3.11 (br. s., 1H), 2.98 (br. s., 1H), 2.56 (s, 3H), 2.48-2.53 (m, 1H), 2.45 (s, 3H), 1.26-1.68 (m, 4H), 1.22 (t, J=7.1 Hz, 3H), 1.18 (s, 9H), 0.97 (br.s., 6H). LCMS (M+H)=493.5.

### EXAMPLE 9

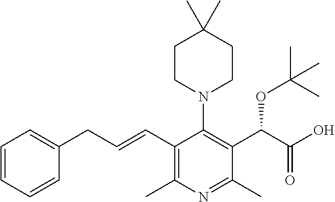 [SMILES: Cc1nc(C)c([C@H](OC(C)(C)C)C(=O)O)c(N2CCC(C)(C)CC2)c1/C=C/Cc1ccccc1]

(S,E)-2-(tert-Butoxy)-2-(4-(4,4-dimethylpiperidin-1-yl)-2,6-dimethyl-5-(3-phenylprop-1-en-1-yl)pyridin-3-yl)acetic acid: A mixture of (S,E)-ethyl 2-(tert-butoxy)-2-(4-(4,4-dimethylpiperidin-1-yl)-2,6-dimethyl-5-(3-phenylprop-1-en-1-yl)pyridin-3-yl)acetate (0.038 g, 0.077 mmol) and LiOH (0.018 g, 0.771 mmol) in 9:1 EtOH/H₂O was refluxed for 2.5 h. Then, cooled and purified by prep-HPLC to afford (S,E)-2-(tert-butoxy)-2-(4-(4,4-dimethylpiperidin-1-yl)-2,6-dimethyl-5-(3-phenylprop-1-en-1-yl)pyridin-3-yl)acetic acid (0.0343 g, 0.074 mmol, 96% yield) as colorless paste. ¹H NMR (500 MHz, CDCl₃) δ 7.31-7.35 (m, 2H), 7.25 (d, J=7.3 Hz, 3H), 6.32 (d, J=16.1 Hz, 1H), 5.79 (td, J=6.6, 16.0 Hz, 1H), 5.68 (br. S., 1H), 3.54-3.67 (m, 4H), 2.70-2.83 (m, 2H), 2.69 (s, 3H), 2.50 (s, 3H), 1.38-1.57 (m, 4H), 1.19 (s, 9H), 0.95 (s, 6H). LCMS (M+H)=465.5.

### EXAMPLE 10

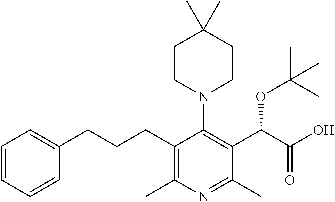 [SMILES: Cc1nc(C)c([C@H](OC(C)(C)C)C(=O)O)c(N2CCC(C)(C)CC2)c1CCCc1ccccc1]

(S)-2-(tert-Butoxy)-2-(4-(4,4-dimethylpiperidin-1-yl)-2,6-dimethyl-5-(3-phenylpropyl)pyridin-3-yl)acetic acid: A mixture of (S,E)-2-(tert-butoxy)-2-(4-(4,4-dimethylpiperidin-1-yl)-2,6-dimethyl-5-(3-phenylprop-1-en-1-yl)pyridin-3-yl)acetic acid (0.0192 g, 0.041 mmol) and 10% Pd/C (4.40 mg, 4.13 μmol) in 1:1 MeOH/EtOAc (5 mL) was left under balloon H₂ atmosphere for 1 h. Then, filtered through a plug of celite and concentrated to give (S)-2-(tert-butoxy)-2-(4-(4,4-dimethylpiperidin-1-yl)-2,6-dimethyl-5-(3-phenylpropyl)pyridin-3-yl)acetic acid (0.019 g, 0.039 mmol, 96% yield) as. ¹H NMR (500 MHz, CDCl₃) δ 7.30-7.34 (m, 2H), 7.20-7.25 (m, 3H), 5.78 (br. s., 1H), 3.22-3.47 (m, 2H), 2.55-3.05 (m, 6H), 2.66 (s, 3H), 2.49 (s, 3H), 1.77-1.94 (m, 2H), 1.26-1.68 (m, 4H), 1.20 (s, 9H), 0.98 (br. s., 6H). LCMS (M+H)=467.5.

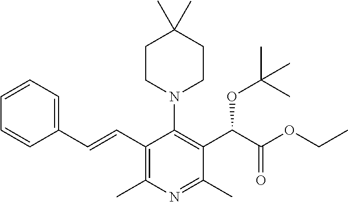 [SMILES: CCOC(=O)[C@@H](OC(C)(C)C)c1c(C)nc(C)c(/C=C/c2ccccc2)c1N1CCC(C)(C)CC1]

(S,E)-Ethyl 2-(tert-butoxy)-2-(4-(4,4-dimethylpiperidin-1-yl)-2,6-dimethyl-5-styrylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.0569 g, 0.125 mmol),(E)-styrylboronic acid (0.028 g, 0.187 mmol) and 2M Na₂CO₃ (0.156 ml, 0.312 mmol) in DMF (3 mL) was degassed for 10 min. Then, Pd(Ph₃P)₄ (0.014 g, 0.012 mmol) added, degassed for 5 min and placed in a pre-heated oil bath at 100° C. After 2 h at 110° C., cooled and purified by prep-HPLC to afford (S,E)-ethyl 2-(tert-butoxy)-2-(4-(4,4-dimethylpiperidin-1-yl)-2,6-dimethyl-5-styrylpyridin-3-yl)acetate (0.0456 g, 0.095 mmol, 76% yield) as pale yellow paste which turned to white foam. ¹H NMR (500 MHz, CDCl₃) δ 7.52-7.57 (m, 2H), 7.39-7.45 (m, 2H), 7.30-7.35 (m, 1H), 7.18 (d, J=16.6 Hz, 1H), 6.53 (d, J=16.6 Hz, 1H), 6.09 (s, 1H), 4.12-4.29 (m, 2H), 2.94-3.61 (m, 4H), 2.60 (s, 3H), 2.53 (s, 3H), 1.29-1.68 (m, 4H), 1.25 (t, J=7.2 Hz, 3H), 1.21 (s, 9H), 0.94 (br.s., 6H). LCMS (M+H)=479.5.

### EXAMPLE 11

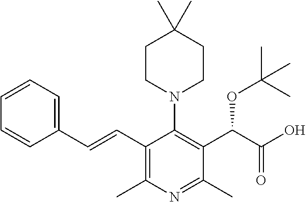 [SMILES: Cc1nc(C)c([C@H](OC(C)(C)C)C(=O)O)c(N2CCC(C)(C)CC2)c1/C=C/c1ccccc1]

(S,E)-2-(tert-Butoxy)-2-(4-(4,4-dimethylpiperidin-1-yl)-2,6-dimethyl-5-styrylpyridin-3-yl)acetic acid: A mixture of (S,E)-ethyl 2-(tert-butoxy)-2-(4-(4,4-dimethylpiperidin-1-yl)-2,6-dimethyl-5-styrylpyridin-3-yl)acetate (0.0406 g, 0.085 mmol) and LiOH (0.020 g, 0.848 mmol) in 9:1 EtOH/H₂O (2 mL) was refluxed for 2.5 h. Then, cooled and purified by prep-HPLC to afford (S,E)-2-(tert-butoxy)-2-(4-(4,4-dimethylpiperidin-1-yl)-2,6-dimethyl-5-styrylpyridin-3-yl)acetic acid (0.035 g, 0.078 mmol, 92% yield) as white solid. ¹H NMR (500 MHz, CDCl₃) δ 7.49-7.53 (m, 2H), 7.41 (t, J=7.6 Hz, 2H), 7.31-7.37 (m, 1H), 7.02 (d, J=16.6 Hz, 1H), 6.50 (d, J=16.6 Hz, 1H), 5.73 (br. S., 1H), 3.64-3.76 (m, 2H), 2.84-2.96 (m, 2H), 2.77 (s, 3H), 2.59 (s, 3H), 1.40-1.59 (m, 4H), 1.23 (s, 9H), 0.92 (s, 6H). LCMS (M+H)=451.5.

### EXAMPLE 12

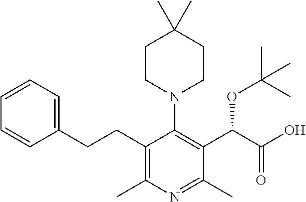 [SMILES: Cc1nc(C)c([C@H](OC(C)(C)C)C(=O)O)c(N2CCC(C)(C)CC2)c1CCc1ccccc1]

(S)-2-(tert-Butoxy)-2-(4-(4,4-dimethylpiperidin-1-yl)-2,6-dimethyl-5-phenethylpyridin-3-yl)acetic acid: A mixture of (S,E)-2-(tert-butoxy)-2-(4-(4,4-dimethylpiperidin-1-yl)-2,6-dimethyl-5-styrylpyridin-3-yl)acetic acid (0.0182 g, 0.040 mmol) and 10% Pd/C (4.30 mg, 4.04 μmol) in 1:1 MeOH/EtOAc (5 mL) was left under balloon H₂ atmosphoere for 1 h. Then, filtered through a plug of celite and concentrated to give (S)-2-(tert-butoxy)-2-(4-(4,4-dimethylpiperidin-1-yl)-2,6-dimethyl-5-phenethylpyridin-3-yl)acetic acid (0.0182 g, 0.039 mmol, 97% yield) as white solid. ¹H NMR (500 MHz, CDCl₃) δ 7.33-7.38 (m, 2H), 7.24-7.28 (m, 3H), 5.84 (br. s., 1H), 3.51-3.63 (m, 2H), 2.77-3.20 (m, 6H), 2.71 (s, 3H), 2.66 (s, 3H), 1.48-1.79 (m, 4H), 1.25 (s, 9H), 1.06 (s, 6H). LCMS (M+H)=453.5.

### EXAMPLE 13

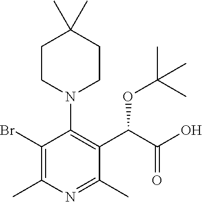 [SMILES: Cc1nc(C)c([C@H](OC(C)(C)C)C(=O)O)c(N2CCC(C)(C)CC2)c1Br]

(S)-2-(5-Bromo-4-(4,4-dimethylpiperidin-1-yl)-2,6-dimethylpyridin-3-yl)-2-(tert-butoxy)acetic acid: (S)-2-(5-Bromo-4-(4,4-dimethylpiperidin-1-yl)-2,6-dimethylpyridin-3-yl)-2-(tert-butoxy)acetic acid: A mixture of (S)-ethyl 2-(5-bromo-4-(4,4-dimethylpiperidin-1-yl)-2,6-dimethylpyridin-3-yl)-2-(tert-butoxy)acetate (0.02 g, 0.044 mmol) and LiOH (10.52 mg, 0.439 mmol) in 9:1 EtOH/H₂O (2 mL) was refluxed for 4 h. Then, cooled and purified by prep-HPLC to afford (S)-2-(5-bromo-4-(4,4-dimethylpiperidin-1-yl)-2,6-dimethylpyridin-3-yl)-2-(tert-butoxy)acetic acid (0.0157 g, 0.037 mmol, 84% yield) as colorless paste. ¹H NMR (500 MHz, CDCl₃) δ 5.97 (br. s., 1H), 4.10 (t, J=11.3 Hz, 1H), 3.54 (br. s., 1H), 3.09 (br. s., 1H), 2.66 (s, 3H), 2.56 (br. s., 4H), 1.68-1.52 (m, 2H), 1.40 (t, J=15.2 Hz, 2H), 1.26 (br. s., 9H), 1.09 (s, 3H), 1.02 (s, 3H). LCMS (M+H)=429.2.

### EXAMPLE 14

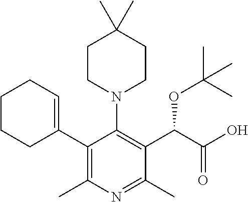 [SMILES: Cc1nc(C)c([C@H](OC(C)(C)C)C(=O)O)c(N2CCC(C)(C)CC2)c1C1=CCCCC1]

(S)-2-(tert-Butoxy)-2-(5-(cyclohex-1-en-1-yl)-4-(4,4-dimethylpiperidin-1-yl)-2,6-dimethylpyridin-3-yl)acetic acid: To a mixture of (S)-ethyl 2-(5-bromo-4-(4,4-dimethylpiperidin-1-yl)-2,6-dimethylpyridin-3-yl)-2-(tert-butoxy)acetate (20 mg, 0.044 mmol), 1-cyclohexen-1-yl boronic acid (6.64 mg, 0.053 mmol) and Cs₂CO₃ (28.6 mg, 0.088 mmol) in 1,4-dioxane (2 mL)/water (0.400 mL) was added Pd(PPh₃)₄ (5.07 mg, 4.39 μmol). The mixture was flushed with nitrogen and then heated at 85° C. for 3 hrs. The mixture was diluted with water and then extracted with EtOAc (2×20 mL). The organic layers were combined, washed with brine and concentrated to give a crude of (S)-ethyl 2-(tert-butoxy)-2-(5-(cyclohex-1-en-1-yl)-4-(4,4-dimethylpiperidin-1-yl)-2,6-dimethylpyridin-3-yl)acetate.

The residue was diluted with MeOH (1 mL), and 1 mL of 1 N NaOH was added. The mixture was heated at 85° C. for 2 hrs. All solvents were removed under vacuum and the residue was purified by prep-HPLC to give (S)-2-(tert-butoxy)-2-(5-(cyclohex-1-en-1-yl)-4-(4,4-dimethylpiperidin-1-yl)-2,6-dimethylpyridin-3-yl)acetic acid (7.1 mg, 0.017 mmol, 37.7% yield). LCMS (M+H)=429.5.

### EXAMPLE 15

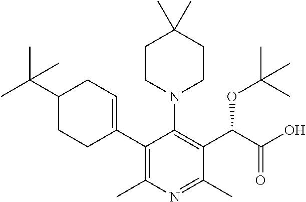 [SMILES: Cc1nc(C)c([C@H](OC(C)(C)C)C(=O)O)c(N2CCC(C)(C)CC2)c1C1=CCC(C(C)(C)C)CC1]

(2S)-2-(tert-Butoxy)-2-(5-(4-(tert-butyl)cyclohex-1-en-1-yl)-4-(4,4-dimethylpiperidin-1-yl)-2,6-dimethylpyridin-3-yl)acetic acid: To a mixture of (S)-ethyl 2-(5-bromo-4-(4,4-dimethylpiperidin-1-yl)-2,6-dimethylpyridin-3-yl)-2-(tert-butoxy)acetate (20 mg, 0.044 mmol), 4-tert-butyl-1-cyclohexen-1-ylboronic acid (8.00 mg, 0.044 mmol) and Cs₂CO₃ (28.6 mg, 0.088 mmol) in 1,4-dioxane (2 mL)/water (0.400 mL) was added Pd(PPh₃)₄ (5.07 mg, 4.39 μmol). The mixture was flushed with nitrogen and then heated at 85° C. for 3 hrs. The mixture was diluted with water and then extracted with EtOAc (2×20 mL). The organic layers were combined, washed with brine and concentrated to give a crude of (2S)-ethyl 2-(tert-butoxy)-2-(5-(4-(tert-butyl)cyclohex-1-en-1-yl)-4-(4,4-dimethylpiperidin-1-yl)-2,6-dimethylpyridin-3-yl)acetate

The residue was diluted with MeOH (1 mL) and 1 mL of 1 N NaOH was added. The mixture was heated at 85° C. for 2 hrs. All solvents were removed under vacuum and the residue was purified by prep-HPLC to give (2S)-2-(tert-butoxy)-2-(5-(4-(tert-butyl)cyclohex-1-en-1-yl)-4-(4,4-dimethylpiperidin-1-yl)-2,6-dimethylpyridin-3-yl)acetic acid (6.1 mg, 0.013 mmol, 28.7% yield). LCMS (M+H)=485.5.

### EXAMPLE 16

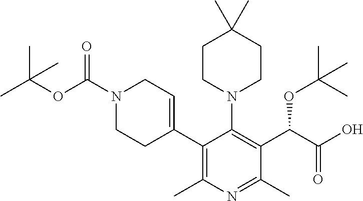 [SMILES: Cc1nc(C)c([C@H](OC(C)(C)C)C(=O)O)c(N2CCC(C)(C)CC2)c1C1=CCN(C(=O)OC(C)(C)C)CC1]

(S)-2-(tert-Butoxy)-2-(1′-(tert-butoxycarbonyl)-4-(4,4-dimethylpiperidin-1-yl)-2,6-dimethyl-1′,2′,3′,6′-tetrahydro-[3,4′-bipyridin]-5-yl)acetic acid: A mixture of (S)-ethyl 2-(5-bromo-4-(4,4-dimethylpiperidin-1-yl)-2,6-dimethylpyridin-3-yl)-2-(tert-butoxy)acetate (50 mg, 0.110 mmol), (1-(tert-butoxycarbonyl)-1,2,3,6-tetrahydropyridin-4-yl)boronic acid (49.9 mg, 0.220 mmol) and 2M Na₂CO₃ (0.137 mL, 0.274 mmol) in 1,4-dioxane (2 mL) was degassed for 10 min. Then, Pd(Ph₃P)₄ (6.34 mg, 5.49 μmol) was added, degassed for 5 min and placed in a pre-heated oil bath at 90° C. After 3 h, cooled, concentrated and purified by prep HPLC to afford desired ester, which was treated with 1NNaOH (0.329 mL, 0.329 mmol) in MeOH (2 mL) at 75° C. for 5 h. Mixture was then cooled and purified by prep HPLC to afford (S)-2-(tert-butoxy)-2-(1′-(tert-butoxycarbonyl)-4-(4,4-dimethylpiperidin-1-yl)-2,6-dimethyl-1′,2′,3′,6′-tetrahydro-[3,4′-bipyridin]-5-yl)acetic acid (17 mg, 0.032 mmol, 29.2% yield). ¹H NMR (500 MHz, DMSO-d₆) δ 5.87 (br. s., 0.7H), 5.73 (br. s., 0.3H), 5.63-5.49 (m, 1H), 4.18 (d, J=18.3 Hz, 1H), 3.98 (d, J=15.8 Hz, 1H), 3.91 (s, 1H), 3.82 (br. s., 1H), 3.72 (br. s., 1H), 3.29 (br. s., 1H), 3.19 (d, J=13.2 Hz, 2H), 2.87-2.80 (m, 1H), 2.46 (br. s., 1H), 2.38 (s, 3H), 2.30 (br. s., 1H), 2.26 (s, 3H), 1.54 (br. s., 1H), 1.47 (br. s., 1H), 1.43 (s, 9H), 1.34-1.18 (m, 2H), 1.12 (s, 9H), 0.94 (br. s., 6H). LCMS (M+H)=530.7.

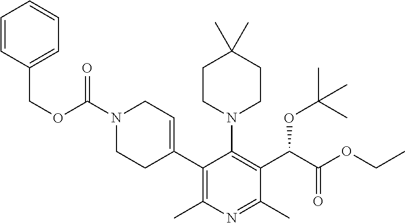 [SMILES: CCOC(=O)[C@@H](OC(C)(C)C)c1c(C)nc(C)c(C2=CCN(C(=O)OCc3ccccc3)CC2)c1N1CCC(C)(C)CC1]

(S)-Benzyl 5-(1-(tert-butoxy)-2-ethoxy-2-oxoethyl)-4-(4,4-dimethylpiperidin-1-yl)-2,6-dimethyl-5,6′-dihydro-[3,4′-bipyridine]-1′(2′H)-carboxylate: A mixture of (S)-ethyl 2-(5-bromo-4-(4,4-dimethylpiperidin-1-yl)-2,6-dimethylpyridin-3-yl)-2-(tert-butoxy)acetate (500 mg, 1.098 mmol), (1-((benzyloxy)carbonyl)-1,2,3,6-tetrahydropyridin-4-yl)boronic acid (573 mg, 2.196 mmol) and 2M Na₂CO₃ (1.372 mL, 2.74 mmol) in 1,4-dioxane (10 mL) was degassed for 10 min. Then, Pd(Ph₃P)₄ (63.4 mg, 0.055 mmol) was added, degassed for 5 min and placed in a pre-heated oil bath at 90° C. After 3 h, cooled, diluted with ether (50 mL), washed with water (20 mL), brine (20 mL), dried (Na₂SO₄), filtered, concentrated and purified by flash chromatography using 5-40% EtOAc/Hex to afford (S)-benzyl 5-(1-(tert-butoxy)-2-ethoxy-2-oxoethyl)-4-(4,4-dimethylpiperidin-1-yl)-2,6-dimethyl-5′,6′-dihydro-[3,4′-bipyridine]-1′(2′H)-carboxylate (440 mg, 0.744 mmol, 67.7% yield) as viscous yellow oil. ¹H NMR (500 MHz, CDCl₃) δ 7.48-7.30 (m, 5H), 6.09 (br. s., 0.6H), 5.97 (s., 0.4H), 5.85 (s., 0.4H), 5.58 (br. s., 0.6H), 5.27-5.13 (m, 2H), 4.32-4.08 (m, 3H), 3.86-3.76 (m, 1H), 3.35 (t, J=11.3 Hz, 1H), 3.15 (d, J=11.2 Hz, 1H), 2.95 (d, J=10.9 Hz, 1H), 2.55 (s, 3H), 2.52 (br. s., 1H), 2.50-2.41 (m, 1H), 2.38 (d, J=2.7 Hz, 3H), 2.07 (s, 1H), 1.62-1.48 (m, 2H), 1.38-1.32 (m, 1H), 1.28 (t, J=7.2 Hz, 2H), 1.24 (t, J=7.1 Hz, 3H), 1.20 (s, 9H), 1.00 (s, 3H), 0.97-0.89 (m, 3H). LCMS (M+H)=592.8.

### EXAMPLE 17 & 18

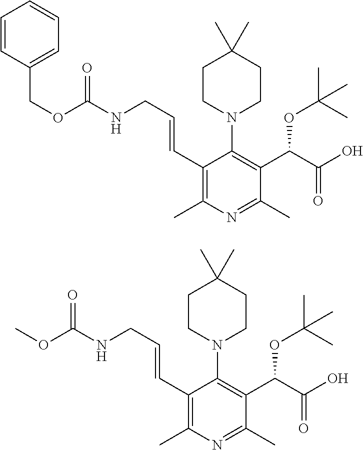

(S,E)-2-(5-(3-(((Benzyloxy)carbonyl)amino)prop-1-en-1-yl)-4-(4,4-dimethylpiperidin-1-yl)-2,6-dimethylpyridin-3-yl)-2-(tert-butoxy)acetic acid (17) & (S,E)-2-(tert-Butoxy)-2-(4-(4,4-dimethylpiperidin-1-yl)-5-(3-((methoxycarbonyl)amino)prop-1-en-1-yl)-2,6-dimethylpyridin-3-yl)acetic acid (18): A mixture of (S)-ethyl 2-(5-bromo-4-(4,4-dimethylpiperidin-1-yl)-2,6-dimethylpyridin-3-yl)-2-(tert-butoxy)acetate (50 mg, 0.110 mmol), (E)-(3-(((benzyloxy)carbonyl)amino)prop-1-en-1-yl)boronic acid (51.6 mg, 0.220 mmol) and 2M Na2CO3 (0.137 mL, 0.274 mmol) in 1,4-dioxane (2 mL) was degassed for 10 min. Then, Pd(Ph₃P)₄ (6.34 mg, 5.49 μmol) was added, degassed for 5 min and placed in a pre-heated oil bath at 90° C. After 3 h, cooled, diluted with ether (50 mL), washed with water (20 mL), brine (20 mL), dried (Na₂SO₄), filtered, concentrated to afford crude, which was tretad with 1N NaOH (0.549 mL, 0.549 mmol) in MeOH (2 mL) at 75° C. for 5 h. Mixture was then cooled and purified by prep HPLC to afford two products. Product 1:(S,E)-2-(5-(3-(((benzyloxy)carbonyl)amino)prop-1-en-1-yl)-4-(4,4-dimethylpiperidin-1-yl)-2,6-dimethylpyridin-3-yl)-2-(tert-butoxy)acetic acid (12 mg, 0.022 mmol, 20.33% yield): ¹H NMR (500 MHz, DMSO-d₆) δ 7.62 (br. s., 1H), 7.43-7.21 (m, 5H), 6.55 (d, J=16.5 Hz, 1H), 5.82 (br. s., 1H), 5.54 (d, J=15.8 Hz, 1H), 5.16-4.94 (m, 2H), 3.84 (d, J=5.1 Hz, 2H), 3.52-3.42 (m, 2H), 2.90 (s, 2H), 2.38 (s, 3H), 2.28 (s, 3H), 1.54 (br. s., 1H), 1.43 (br. s., 1H), 1.23 (br. s., 2H), 1.11 (s, 9H), 0.94 (br. s., 6H). LCMS (M+H)=538.3; and (S,E)-2-(tert-butoxy)-2-(4-(4,4-dimethylpiperidin-1-yl)-5-(3-((methoxycarbonyl)amino)prop-1-en-1-yl)-2,6-dimethylpyridin-3-yl)acetic acid (16.5 mg, 0.036 mmol, 32.6% yield): ¹H NMR (500 MHz, DMSO-d₆) δ 7.47 (br. s., 1H), 6.55 (d, J=16.1 Hz, 1H), 5.81 (br. s., 1H), 5.54 (dt, J=16.0, 5.4 Hz, 1H), 3.82 (q, J=15.9 Hz, 2H), 3.53-3.33 (m, 5H), 2.90 (s, 2H), 2.38 (s, 3H), 2.30 (s, 3H), 1.55 (br. s., 1H), 1.46 (br. s., 1H), 1.33 (br. s., 1H), 1.27 (br. s., 1H), 1.11 (s, 9H), 0.95 (s, 6H). LCMS (M+H)=462.3.

### EXAMPLE 19 & 20

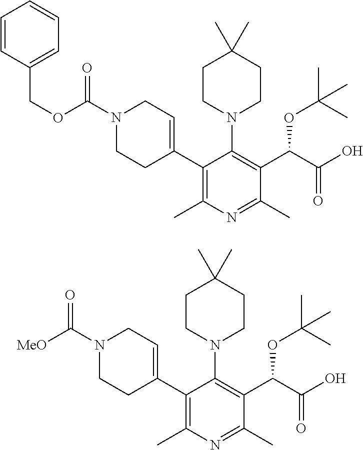

(S)-2-(1′-((Benzyloxy)carbonyl)-4-(4,4-dimethylpiperidin-1-yl)-2,6-dimethyl-1′,2′,3′,6′-tetrahydro-[3,4′-bipyridin]-5-yl)-2-(tert-butoxy)acetic acid (19) & (S)-2-(tert-Butoxy)-2-(4-(4,4-dimethylpiperidin-1-yl)-1′-(methoxycarbonyl)-2,6-dimethyl-1′,2′,3′,6′-tetrahydro-[3,4′-bipyridin]-5-yl)acetic acid (20): To a solution of (S)-benzyl 5-(1-(tert-butoxy)-2-ethoxy-2-oxoethyl)-4-(4,4-dimethylpiperidin-1-yl)-2,6-dimethyl-5′,6′-dihydro-[3,4′-bipyridine]-1′(2′H)-carboxylate (30 mg, 0.051 mmol) in MeOH (1 mL) was added 1N NaOH (0.253 mL, 0.253 mmol) and the resulting mixture was heated at 75° C. for 5 h. Mixture was then cooled and purified by prep HPLC to afford two compunds. (S)-2-(1′-((benzyloxy)carbonyl)-4-(4,4-dimethylpiperidin-1-yl)-2,6-dimethyl-1′,2′,3′,6′-tetrahydro-[3,4′-bipyridin]-5-yl)-2-(tert-butoxy)acetic acid (12.9 mg, 0.023 mmol, 45.1% yield): ¹H NMR (500 MHz, DMSO-d₆) δ 7.45-7.25 (m, 5H), 5.86 (br. s., 0.7H), 5.72 (br. s., 0.3H), 5.59 (br. s., 1H), 5.13 (br. s., 2H), 4.20 (br. s., 1H), 4.05 (br. s., 1H), 3.83 (br. s., 1H), 3.43-3.18 (m, 4H), 2.88-2.82 (m, 1H), 2.46 (br. s., 1H), 2.40-2.30 (m, 4H), 2.25 (br. s., 3H), 1.53 (br. s., 1H), 1.44 (br. s., 1H), 1.24 (br. s., 2H), 1.11 (s, 9H), 0.93 (br. s., 3H), 0.85 (br. s., 3H). LCMS (M+H)=564.3; and (S)-2-(tert-butoxy)-2-(4-(4,4-dimethylpiperidin-1-yl)-1′-(methoxycarbonyl)-2,6-dimethyl-1′,2′,3′,6′-tetrahydro-[3,4′-bipyridin]-5-yl)acetic acid (7.6 mg, 0.016 mmol, 30.7% yield): ¹H NMR (500 MHz, DMSO-d₆) δ 5.84 (br. s., 0.7H), 5.69 (br. s., 0.3H), 5.58 (br. s., 1H), 4.11 (br. s., 1H), 4.07-3.93 (m, 2H), 3.73 (br. s., 1H), 3.31-3.20 (m, 3H), 3.18 (br. s., 1H), 2.95-2.82 (m, 2H), 2.47 (br. s., 1H), 2.42-2.36 (m, 2H), 2.26 (s, 3H), 1.91 (s, 3H), 1.54 (br. s., 1H), 1.45 (d, J=10.6 Hz, 1H), 1.33 (d, J=11.7 Hz, 1H), 1.24 (br. s., 1H), 1.11 (s, 9H), 0.97-0.89 (m, 6H). LCMS (M+H)=488.3.

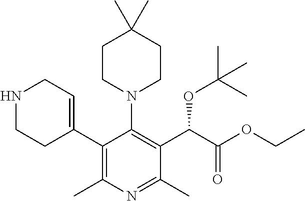 [SMILES: CCOC(=O)[C@@H](OC(C)(C)C)c1c(C)nc(C)c(C2=CCNCC2)c1N1CCC(C)(C)CC1]

(S)-Ethyl 2-(tert-butoxy)-2-(4-(4,4-dimethylpiperidin-1-yl)-2,6-dimethyl-1′,2′,3′,6′-tetrahydro-[3,4′-bipyridin]-5-yl)acetate: A solution of (S)-benzyl 5-(1-(tert-butoxy)-2-ethoxy-2-oxoethyl)-4-(4,4-dimethylpiperidin-1-yl)-2,6-dimethyl-5′,6′-dihydro-[3,4′-bipyridine]-1′(2′H)-carboxylate (200 mg, 0.338 mmol) in methanol (2 mL)/ethyl acetate (2 mL) was added Pd(OH)₂ (83 mg, 0.118 mmol) and the resulting mixture was allowed to stirr under an atmosphere of H₂ for 16 h. At this point mixture was filtered through a pad of celite and concentrated to afford (S)-ethyl 2-(tert-butoxy)-2-(4-(4,4-dimethylpiperidin-1-yl)-2,6-dimethyl-1′,2′,3′,6′-tetrahydro-[3,4′-bipyridin]-5-yl)acetate (150 mg, 0.328 mmol, 97% yield) as viscous oil. Used a sis in the next step without further purification. LCMS (M+H)=458.6.

### EXAMPLE 21

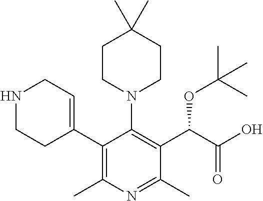 [SMILES: Cc1nc(C)c([C@H](OC(C)(C)C)C(=O)O)c(N2CCC(C)(C)CC2)c1C1=CCNCC1]

(S)-2-(tert-Butoxy)-2-(4-(4,4-dimethylpiperidin-1-yl)-2,6-dimethyl-1′,2′,3′,6′-tetrahydro-[3,4′-bipyridin]-5-yl)acetic acid: To a solution of (S)-ethyl 2-(tert-butoxy)-2-(4-(4,4-dimethylpiperidin-1-yl)-2,6-dimethyl-1′,2′,3′,6′-tetrahydro-[3,4′-bipyridin]-5-yl)acetate (10 mg, 0.022 mmol) in MeOH (1 mL) was added 1N NaOH (0.219 mL, 0.219 mmol) and the resulting mixture was heated at 75° C. for 5 h. Mixture was then cooled and purified by prep HPLC to afford (S)-2-(tert-butoxy)-2-(4-(4,4-dimethylpiperidin-1-yl)-2,6-dimethyl-1′,2′,3′,6′-tetrahydro-[3,4′-bipyridin]-5-yl)acetic acid (3.8 mg, 8.85 μmol, 40.5% yield). ¹H NMR (500 MHz, DMSO-d₆) δ 5.85 (br. s., 0.7H), 5.67 (br. s., 0.3H), 5.55 (br. s., 1H), 3.55-3.40 (m, 6H), 3.33-3.15 (m, 2H), 3.12 (br. s., 1H), 3.01 (d, J=7.3 Hz, 1H), 2.85 (br. s., 1H), 2.40-2.34 (m, 3H), 2.31-2.24 (m, 3H), 1.54 (br. s., 1H), 1.47 (br. s., 1H), 1.36 (br. s., 1H), 1.24 (br. s., 1H), 1.16-1.03 (m, 9H), 0.95 (br. s., 6H). LCMS (M+H)=430.7.

### EXAMPLE 22

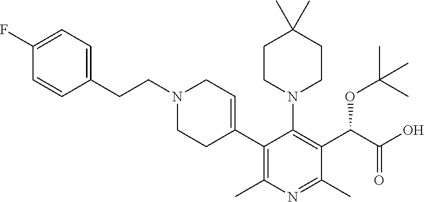 [SMILES: Cc1nc(C)c([C@H](OC(C)(C)C)C(=O)O)c(N2CCC(C)(C)CC2)c1C1=CCN(CCc2ccc(F)cc2)CC1]

(S)-2-(tert-Butoxy)-2-(4-(4,4-dimethylpiperidin-1-yl)-1′-(4-fluorophenethyl)-2,6-dimethyl-1′,2′,3′,6′-tetrahydro-[3,4′-bipyridin]-5-yl)acetic acid: To a stirred solution of (S)-ethyl 2-(tert-butoxy)-2-(4-(4,4-dimethylpiperidin-1-yl)-2,6-dimethyl-1′,2′,3′,6′-tetrahydro-[3,4′-bipyridin]-5-yl)acetate (40 mg, 0.087 mmol) and 2-(4-fluorophenyl)acetaldehyde (24.15 mg, 0.175 mmol) in MeOH (2 mL) was added at once NaCNBH₄ (8.24 mg, 0.131 mmol) and ZnCl₂ (8.93 mg, 0.066 mmol) at rt and the mixture was stirred for 3 h. At this point LCMS indicated completion of raction. 1N NaOH (0.874 mL, 0.874 mmol) was then added and the mixture was heated at 75° C. for 5 h. Mixture was then cooled and purified by prep HPLC to afford (S)-2-(tert-butoxy)-2-(4-(4,4-dimethylpiperidin-1-yl)-1′-(4-fluorophenethyl)-2,6-dimethyl-1′,2′,3′,6′-tetrahydro-[3,4′-bipyridin]-5-yl)acetic acid (35 mg, 0.063 mmol, 72.6% yield). ¹H NMR (500 MHz, DMSO-d₆) δ 7.37-7.25 (m, 2H), 7.09 (t, J=8.8 Hz, 2H), 5.90 (br. s., 0.6H), 5.75 (br. s., 0.3H), 5.54 (br. s., 1H), 3.45-3.40 (m, 2H), 3.16 (br. s., 3H), 2.90 (s, 1H), 2.83-2.77 (m, 2H), 2.72-2.57 (m, 4H), 2.49-2.43 (m, 1H), 2.38 (s, 3H), 2.27 (s, 3H), 2.21 (br. s., 1H), 1.59-1.41 (m, 2H), 1.35 (br. s., 1H), 1.23 (d, J=12.1 Hz, 1H), 1.12 (s, 9H), 0.95 (br. s., 3H), 0.89 (s, 3H). LCMS (M+H)=552.4.

### EXAMPLE 23

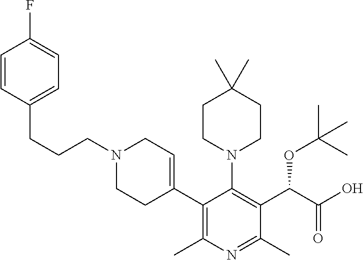 [SMILES: Cc1nc(C)c([C@H](OC(C)(C)C)C(=O)O)c(N2CCC(C)(C)CC2)c1C1=CCN(CCCc2ccc(F)cc2)CC1]

(S)-2-(tert-Butoxy)-2-(4-(4,4-dimethylpiperidin-1-yl)-1′-(3-(4-fluorophenyl)propyl)-2,6-dimethyl-1′,2′,3′,6′-tetrahydro-[3,4′-bipyridin]-5-yl)acetic acid: To a stirred solution of (S)-ethyl 2-(tert-butoxy)-2-(4-(4,4-dimethylpiperidin-1-yl)-2,6-dimethyl-1′,2′,3′,6′-tetrahydro-[3,4′-bipyridin]-5-yl)acetate (40 mg, 0.087 mmol) and 3-(4-fluorophenyl)propanal (26.6 mg, 0.175 mmol) in MeOH (2 mL) was added at once NaCNBH₄ (8.24 mg, 0.131 mmol) and ZnCl₂ (8.93 mg, 0.066 mmol) at rt and the mixture was stirred for 3 h. At this point LCMS indicated completion of raction. 1N NaOH (0.874 mL, 0.874 mmol) was then added and the mixture was heated at 75° C. for 5 h. Mixture was then cooled and purified by prep HPLC to afford (S)-2-(tert-butoxy)-2-(4-(4,4-dimethylpiperidin-1-yl)-1′-(3-(4-fluorophenyl)propyl)-2,6-dimethyl-1′,2′,3′,6′-tetrahydro-[3,4′-bipyridin]-5-yl)acetic acid (16.1 mg, 0.028 mmol, 32.6% yield). ¹H NMR (500 MHz, DMSO-d₆) δ 7.28-7.16 (m, 2H), 7.09 (t, J=8.6 Hz, 2H), 5.85 (br. s., 0.7H), 5.69 (br. s., 0.3H), 5.53 (br. s., 1H), 3.25 (br. s., 1H), 3.18-3.09 (m, 1H), 3.09-2.94 (m, 2H), 2.90-2.81 (m, 1H), 2.63 (d, J=4.4 Hz, 4H), 2.51 (br. s., 3H), 2.40-2.34 (m, 5H), 2.26 (s, 3H), 1.77 (t, J=7.2 Hz, 2H), 1.62-1.40 (m, 2H), 1.35 (br. s., 1H), 1.23 (br. s., 1H), 1.11 (s, 9H), 0.93 (br. s., 6H). LCMS (M+H)=566.3.

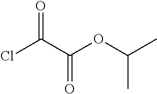

Isopropyl 2-chloro-2-oxoacetate: The propan-2-ol (38.2 mL, 499 mmol) was added drop wise over 15 min to a cold (0° C.), nitrogen purged solution of oxalyl dichloride (101 g, 799 mmol) and the reaction was stirred at room temperature for 2.5 h. Then a reflux condenser was fitted and a slight vacuum was applied for about 1 h until HCl gas was removed (the HCl was trapped in by a sat'd solution of NaHCO₃). The reflux condenser was removed and the flask was fitted with a short path distillation head. Excess reagent was removed by distillation under house vacuum (oil bath heated to 65° C.), and then the temperature was raised to between 85-95° C. and the product was distilled (NOTE: The 1^{st} fraction of ˜5 mL was discarded) to provide isopropyl 2-chloro-2-oxoacetate 52.62 g (70%).

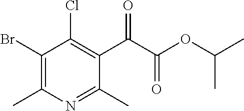 [SMILES: Cc1nc(C)c(C(=O)C(=O)OC(C)C)c(Cl)c1Br]

Isopropyl 2-(5-bromo-4-chloro-2,6-dimethylpyridin-3-yl)-2-oxoacetate: A solution of 2M isopropyl magnesium chloride (84 mL, 168 mmol) was added drop wise over 20 min to a cold (−70° C.), nitrogen purged solution of 3,5-dibromo-4-chloro-2,6-dimethylpyridine (48 g, 160 mmol) and copper(I)bromide-dimethyl sulfide complex (1.65 g, 8.02 mmol) in THF (240 mL), which was then allowed to warm to −10° C. over 60 min. The reaction mixture was transferred via cannula into a 1 L RB-flask containing isopropyl 2-chloro-2-oxoacetate (26.6 g, 176 mmol) in THF (160 mL) maintained at −60° C., and the reaction stirred an additional 2.5 h while being allowed to warm to −10° C. The reaction was quenched upon diluted with a mixture of 10% NH₄Cl solution (80 mL) in ether (320 mL). The organic layer was washed with 160 mL of sat'd NaHCO₃/10% NH₄Cl solution (1:1), brine, and dried (Na₂SO₄). The crude product was charged (DCM solution) to a 330 g ISCO silica gel cartridge and gradient eluted (5-20% EtOAc/hexanes) using an Isolera chromatography station gave isopropyl 2-(5-bromo-4-chloro-2,6-dimethylpyridin-3-yl)-2-oxoacetate 40.38 g (76%). ¹H NMR (500 MHz, CDCl₃) δ 5.28-5.21 (m, 1H), 2.77 (s, 3H), 2.47 (s, 3H), 1.40 (d, J=6.3 Hz, 6H). LCMS (M+H)=336.04.

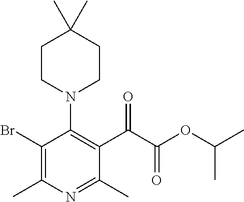 [SMILES: Cc1nc(C)c(C(=O)C(=O)OC(C)C)c(N2CCC(C)(C)CC2)c1Br]

Isopropyl 2-(5-bromo-4-(4,4-dimethylpiperidin-1-yl)-2,6-dimethylpyridin-3-yl)-2-oxoacetate: To a stirred solution of isopropyl 2-(5-bromo-4-chloro-2,6-dimethylpyridin-3-yl)-2-oxoacetate (7.2 g, 21.52 mmol) and DIEA (4.13 mL, 23.67 mmol) in anhydrous acetonitrile (15 mL) was added 4,4-dimethylpiperidine (2.68 g, 23.67 mmol) in acetonitrile (15 mL). The resulting solution was placed in a pre-heated oil bath at 75° C. After heating (75-78° C.) for 24 h and the temperature was raised to 85° C. for 24 h. Another portion of DIEA (3.5 mL, 20.04 mmol) and 4,4-dimethylpiperidine (0.27 g, 2.4 mmol) in acetonitrile (3 mL) was added and hearted at 85° C. for a day. The reaction mixture was diluted with ether (100 mL), washed with water (100 mL), brine (50 mL), dried (MgSO₄), filtered, concentrated and purified by ISCO 120 g cartridge (EtOAc/hex: 0 to 20%) to afford isopropyl 2-(5-bromo-4-(4,4-dimethylpiperidin-1-yl)-2,6-dimethylpyridin-3-yl)-2-oxoacetate (6.8 g, 16.53 mmol, 77% yield. ¹H NMR (500 MHz, CDCl₃) δ 5.25-5.11 (m, 1H), 3.17 (br. s., 4H), 2.71 (s, 3H), 2.41 (s, 3H), 1.42-1.37 (m, 10H), 1.00 (s, 6H).). LCMS (M+H)=413.3.

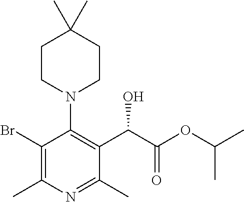 [SMILES: Cc1nc(C)c([C@H](O)C(=O)OC(C)C)c(N2CCC(C)(C)CC2)c1Br]

(S)-Isopropyl 2-(5-bromo-4-(4,4-dimethylpiperidin-1-yl)-2,6-dimethylpyridin-3-yl)-2-hydroxyacetate: To a yellow solution of isopropyl 2-(5-bromo-4-(4,4-dimethylpiperidin-1-yl)-2,6-dimethylpyridin-3-yl)-2-oxoacetate (7.7 g, 18.72 mmol) and (R)-1-methyl-3,3-diphenylhexahydropyrrolo[1,2-c][1,3,2]oxazaborole (7.5 mL, 7.50 mmol) in anhydrous toluene (100 mL) was added drop wise 50% catecholborane/toluene (6 mL, 28.0 mmol) over 5 min at −50° C. Then, the reaction mixture was slowly warmed to −30° C. over 1 h and left in refrigerator (−20° C.) for 3 days. Then, the reaction mixture was diluted with EtOAc (100 mL) and 20 mL of 1M Na₂CO₃, and vigorously stirred for 30 min. Aqueous layer separated and organic layer washed with sat'd Na₂CO₃ (2×25 mL) by vigorously stirring for 15 each time, then dried (MgSO₄), filtered and concentrated to give crude product as light purple paste which was purified by flash chromatography using 0 to 40% EtOAc/hex to afford (S)-isopropyl 2-(5-bromo-4-(4,4-dimethylpiperidin-1-yl)-2,6-dimethylpyridin-3-yl)-2-hydroxyacetate (6.7 g, 15.72 mmol, 84% yield) as colorless thick paste. ¹H NMR (500 MHz, CDCl₃) δ 5.85 (d, J=5.7 Hz, 1H), 5.59 (d, J=7.4 Hz, 1H), 5.08 (dt, J=12.5, 6.3 Hz, 1H), 3.98-3.88 (m, 1H), 3.88-3.78 (m, 1H), 2.76-2.68 (m, 1H), 2.67 (s, 3H), 2.64-2.58 (m, 1H), 2.57 (s, 3H), 1.73 (td, J=12.8, 4.8 Hz, 1H), 1.65-1.59 (m, 1H), 1.47-1.35 (m, 2H), 1.27 (d, J=6.3 Hz, 3H), 1.17 (d, J=6.1 Hz, 3H), 1.09 (s, 3H), 1.04 (s, 3H). LCMS (M+H)=414.6.

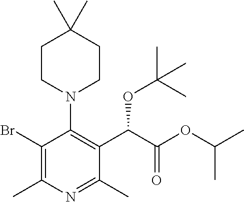 [SMILES: Cc1nc(C)c([C@H](OC(C)(C)C)C(=O)OC(C)C)c(N2CCC(C)(C)CC2)c1Br]

(S)-Isopropyl 2-(5-bromo-4-(4,4-dimethylpiperidin-1-yl)-2,6-dimethylpyridin-3-yl)-2-(tert-butoxy)acetate: A stirred ice-cold yellow mixture of (S)-isopropyl 2-(5-bromo-4-(4,4-dimethylpiperidin-1-yl)-2,6-dimethylpyridin-3-yl)-2-hydroxyacetate (6.7 g, 16.21 mmol) and 70% HClO₄ (2.2 mL, 25.6 mmol) in dichloromethane (400 mL) was saturated with isobutylene gas by bubbling through the reaction mixture (10 min). The reaction mixture was cloudy sealed in a seal tube, stirred for 24 h at rt. The reaction mixture was recooled in a −10° C. bath, bubbled additional isobutylene (˜15 min). The reaction mixture became a clear solution at this point. The tube was sealed and stirred at rt for 16 h. LCMs at this point showed incomplete reaction. So, the reaction mixture was cooled down to −30° C. and bubbled isobutene (˜15 min). After 24 h, reaction mixture was neutralized with sat. Na₂CO₃ (20 mL), organic layer separated and aqueous layer was extracted with CH₂Cl₂ (25 mL). The combined organic layers were dried (MgSO₄), filtered, concentrated and purified on a ISCO 120 g column (EtOAc/hex: 0 to 40%) to afford (S)-isopropyl 2-(5-bromo-4-(4,4-dimethylpiperidin-1-yl)-2,6-dimethylpyridin-3-yl)-2-(tert-butoxy)acetate (5.43 g, 9.83 mmol, 60.7% yield) as a viscous oil. ¹H NMR (500 MHz, CDCl₃) δ 6.26 (br. s., 1H), 5.09-4.97 (m, 1H), 4.06 (br. s., 1H), 3.51 (br. s., 1H), 2.90 (br. s., 1H), 2.65 (s, 3H), 2.56 (s, 3H), 1.72-1.54 (m, 3H), 1.47 (br. s., 1H), 1.37 (br. s., 1H), 1.23-1.20 (m, 12H), 1.15 (d, J=6.1 Hz, 3H), 1.09 (br. s., 3H), 1.04 (br. s., 3H). LCMS (M+H)=471.3.

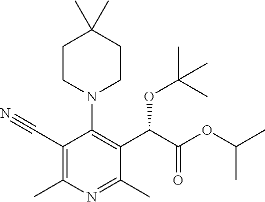 [SMILES: Cc1nc(C)c([C@H](OC(C)(C)C)C(=O)OC(C)C)c(N2CCC(C)(C)CC2)c1C#N]

(S)-Isopropyl 2-(tert-butoxy)-2-(5-cyano-4-(4,4-dimethylpiperidin-1-yl)-2,6-dimethylpyridin-3-yl)acetate: A mixture of CuCN (0.382 g, 4.26 mmol) and (S)-isopropyl 2-(5-bromo-4-(4,4-dimethylpiperidin-1-yl)-2,6-dimethylpyridin-3-yl)-2-(tert-butoxy)acetate (1 g, 2.130 mmol) in DMF (6 mL) was heated in a 160-165° C. for 15 h. The reaction mixture was cooled and filtered off the solid. The filtration was diluted with EtOAc, washed with satd. Na₂CO₃, brine ,dried (MgSO₄), concentrated and purified on a 40 g silica gel cartridge (EtOAc/hex: 5 to 100% to afford (S)-isopropyl 2-(tert-butoxy)-2-(5-cyano-4-(4,4-dimethylpiperidin-1-yl)-2,6-dimethylpyridin-3-yl)acetate (0.6 g, 1.444 mmol, 67.8% yield). ¹H NMR (500 MHz,CDCl₃) δ 5.91 (br. s., 1H), 5.07 (spt, J=6.3 Hz, 1H), 2.71 (s, 3H), 2.61 (s, 3H), 1.23 (d, J=6.3 Hz, 3H), 1.21-1.16 (m, 12H), 1.08 (s, 6H).

### EXAMPLE 24

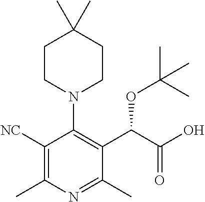 [SMILES: Cc1nc(C)c([C@H](OC(C)(C)C)C(=O)O)c(N2CCC(C)(C)CC2)c1C#N]

(S)-2-(tert-Butoxy)-2-(5-cyano-4-(4,4-dimethylpiperidin-1-yl)-2,6-dimethylpyridin-3-yl)acetic acid: KOH(0.027 g, 0.481 mmol) was added to a solution of (S)-isopropyl 2-(tert-butoxy)-2-(5-cyano-4-(4,4-dimethylpiperidin-1-yl)-2,6-dimethylpyridin-3-yl)acetate (0.02 g, 0.048 mmol) in ethanol (2 mL). The mixture was stirred at 80° C. for 4 h. The reaction mixture was cooled and submitted purified by prep-HPLC to afford (S)-2-(tert-butoxy)-2-(5-cyano-4-(4,4-dimethylpiperidin-1-yl)-2,6-dimethylpyridin-3-yl)acetic acid (0.0139 g, 0.037 mmol, 77% yield). ¹H NMR (500 MHz, DMSO-d₆) δ 3.57 (br. s., 2H), 2.96 (br. s., 2H), 2.56 (s, 3H), 2.48 (s, 4H), 1.53 (br. s., 2H), 1.43 (br. s., 2H), 1.11 (s, 9H), 1.01 (s, 6H). LCMS (M+H): 374.24.

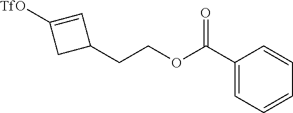 [SMILES: O=C(OCCC1C=C(OS(=O)(=O)C(F)(F)F)C1)c1ccccc1]

2-(3-(((Trifluoromethyl)sulfonyl)oxy)cyclobut-2-en-1-yl)ethyl benzoate: To a 100 mL flame-dried RB-flask was placed 2-(3-oxocyclobutyl)ethyl benzoate (1 g, 4.58 mmol, Ramnauth, J and Lee-Ruff, E.; Canadian Journal Chemistry 2001, 79, 114-120) and 1,1,1-trifluoro-N-phenyl-N-((trifluoromethyl)sulfonyl)methanesulfonamide (1.801 g, 5.04 mmol), followed by THF (10 mL). The solution was chilled to −78° C. under nitrogen. To this was added potassium bis(trimethylsilyl)amide/THF (4.8 mL, 4.80 mmol) dropwise and stirring continued for three hours at −78° C. The cold reaction mixture was quenched with solutions of (1) 2 mL half saturated ammonium chloride solution and 3 mL 0.5 N HCl. The organic residues were extracted into ethyl acetate. The concentrated organic layers was purified on a Biotage 40-gm silica gel column (EtOAc/hex: 0 to 20%) to afford 2-(3-(((trifluoromethyl)sulfonyl)oxy)cyclobut-2-en-1-yl)ethyl benzoate (0.75 g, 2.141 mmol, 46.7% yield). ¹H NMR (400 MHz, CDCl₃) δ 8.09-8.00 (m, 2H), 7.59 (t, J=7.4 Hz, 1H), 7.47 (t, J=7.7 Hz, 2H), 5.57 (s, 1H), 4.46-4.33 (m, 2H), 3.13 (dd, J=13.4, 4.1 Hz, 1H), 2.81-2.69 (m, 1H), 2.58 (d, J=13.6 Hz, 1H), 1.99 (q, J=6.5 Hz, 2H).

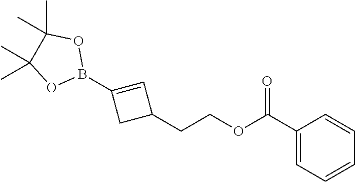 [SMILES: CC1(C)OB(C2=CC(CCOC(=O)c3ccccc3)C2)OC1(C)C]

2-(3-(4,4,5,5-tetramethyl-1,3,2-dioxaborolan-2-yl)cyclobut-2-en-1-yl)ethyl benzoate: 2-(3-(4,4,5,5-Tetramethyl-1,3,2-dioxaborolan-2-yl)cyclobut-2-en-1-yl)ethyl benzoate: 2-(3-(((Trifluoromethyl)sulfonyl)oxy)cyclobut-2-en-1-yl)ethyl benzoate (0.75 g, 2.141 mmol) was mixed with 4,4,4′,4′,5,5,5′,5′-octamethyl-2,2′-bi(1,3,2-dioxaborolane) (0.652 g, 2.57 mmol); potassium acetate (0.504 g, 5.14 mmol) and 1,1′-bis(diphenylphosphino)ferrocene-palladium(II)dichloride CH₂Cl₂ complex (0.087 g, 0.107 mmol). 1,4-Dioxane (12 mL) were added afterward forming a bright orange mixture. It was quickly immersed into a dry-ice bath such that the contents were flash-frozen into solid. Standard evacuation-purge cycles were repeated 4 times under house vacuum and nitrogen was introduced afterward. The solid was allow to melt, forming a bright orange color solution at rt. It was immersed into an oil bath at 70° C., and stirred for 18 h. The mixture was concentrated and purified on a 40 g silica gel cartridge (EtOAc/hexanes: 0 to 40%) to afford 2-(3-(4,4,5,5-tetramethyl-1,3,2-dioxaborolan-2-yl)cyclobut-2-en-1-yl)ethyl benzoate (0.13 g, 0.396 mmol, 18.50% yield). ¹H NMR (500 MHz, CDCl₃) δ 8.16-8.00 (m, 2H), 7.63-7.54 (m, 1H), 7.51-7.40 (m, 2H), 6.97 (d, J=0.8 Hz, 1H), 4.38 (t, J=6.5 Hz, 2H), 3.12-3.03 (m, 1H), 2.84 (dd, J=13.3, 4.3 Hz, 1H), 2.31 (dd, J=13.3, 1.7 Hz, 1H), 2.05-1.92 (m, 2H), 1.29 (s, 12H).

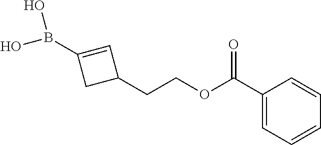 [SMILES: O=C(OCCC1C=C(B(O)O)C1)c1ccccc1]

(3-(2-(Benzoyloxy)ethyl)cyclobut-1-en-1-yl)boronic acid: To a solution of 2-(3-(4,4,5,5-tetramethyl-1,3,2-dioxaborolan-2-yl)cyclobut-2-en-1-yl)ethyl benzoate (0.13 g, 0.396 mmol) in Acetone (10 mL)/water (5.00 mL) was added NaIO₄ (0.424 g, 1.980 mmol) and NH₄OAc (0.153 g, 1.980 mmol) and the resulting mixture was stirred at rt for 16 h. 1N HCl (1 mL) was added and the mixture was stirred for 1 h. The mixture was then diluted with EtOAc (100 mL) and washed with brine (50 mL), dried (Na₂SO₄), filtered and concentrated to afford (3-(2-(benzoyloxy)ethyl)cyclobut-1-en-1-yl)boronic acid (0.097 g, 0.396 mmol, 100% yield) as yellow solid which was used in the next step without purification.

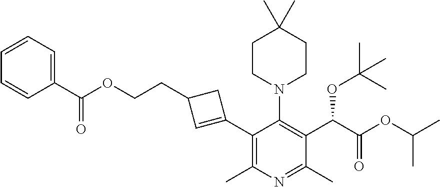 [SMILES: Cc1nc(C)c([C@H](OC(C)(C)C)C(=O)OC(C)C)c(N2CCC(C)(C)CC2)c1C1=CC(CCOC(=O)c2ccccc2)C1]

2-(3-(5-((S)-1-(tert-Butoxy)-2-isopropoxy-2-oxoethyl)-4-(4,4-dimethylpiperidin-1-yl)-2,6-dimethylpyridin-3-yl)cyclobut-2-en-1-yl)ethyl benzoate: A mixture of (S)-isopropyl 2-(5-bromo-4-(4,4-dimethylpiperidin-1-yl)-2,6-dimethylpyridin-3-yl)-2-(tert-butoxy)acetate (0.1 g, 0.213 mmol), (3-(2-(benzoyloxy)ethyl)cyclobut-1-en-1-yl)boronic acid (0.09 g, 0.366 mmol) and Na₂CO₃ (0.023 g, 0.213 mmol) in 1,4-dioxane was digassed and refiled N₂ back (3×). Pd(Ph₃P)₄ (0.025 g, 0.022 mmol) was added and degassed refilled N₂ back. The mixture was stirred at 90° C. for 18 h. The mixture was cooled and diluted with EtOAc, washed with water, brine, dried (MgSO₄), concentrated and purified by prep HPLC to afford 2-(3-(5-((S)-1-(tert-butoxy)-2-isopropoxy-2-oxoethyl)-4-(4,4-dimethylpiperidin-1-yl)-2,6-dimethylpyridin-3-yl)cyclobut-2-en-1-yl)ethyl benzoate (0.053 g, 0.090 mmol, 42.1% yield). LCMS (M+H): 591.7.

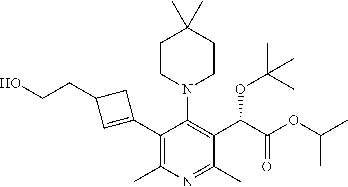 [SMILES: Cc1nc(C)c([C@H](OC(C)(C)C)C(=O)OC(C)C)c(N2CCC(C)(C)CC2)c1C1=CC(CCO)C1]

(2S)-Isopropyl 2-(tert-butoxy)-2-(4-(4,4-dimethylpiperidin-1-yl)-5-(3-(2-hydroxyethyl)cyclobut-1-en-1-yl)-2,6-dimethylpyridin-3-yl)acetate: A solution of 2-(3-(5-((S)-1-(tert-butoxy)-2-isopropoxy-2-oxoethyl)-4-(4,4-dimethylpiperidin-1-yl)-2,6-dimethylpyridin-3-yl)cyclobut-2-en-1-yl)ethyl benzoate (0.053 g, 0.090 mmol) and K₂CO₃ (0.090 mL, 0.179 mmol) in MeOH (1 mL) was stirred at rt for 20 h. The mixture was diluted with EtOAc and washed with 1 N NaOH, brine, dried (Na₂SO₄), concentrated to afford a solid which was purified by flash chromatogarphy (EtOAc/herx: 0 to 25%) to afford (2S)-isopropyl 2-(tert-butoxy)-2-(4-(4,4-dimethylpiperidin-1-yl)-5-(3-(2-hydroxyethyl)cyclobut-1-en-1-yl)-2,6-dimethylpyridin-3-yl)acetate (0.042 g, 0.086 mmol, 96% yield). LCMS (M+H): 487.6.

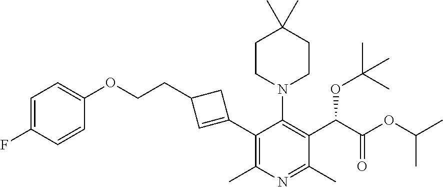 [SMILES: Cc1nc(C)c([C@H](OC(C)(C)C)C(=O)OC(C)C)c(N2CCC(C)(C)CC2)c1C1=CC(CCOc2ccc(F)cc2)C1]

(2S)-Isopropyl 2-(tert-butoxy)-2-(4-(4,4-dimethylpiperidin-1-yl)-5-(3-(2-(4-fluorophenoxy)ethyl)cyclobut-1-en-1-yl)-2,6-dimethylpyridin-3-yl)acetate: To a solution of 4-fluorophenol (0.042 g, 0.378 mmol) and (2S)-isopropyl 2-(tert-butoxy)-2-(4-(4,4-dimethylpiperidin-1-yl)-5-(3-(2-hydroxyethyl)cyclobut-1-en-1-yl)-2,6-dimethylpyridin-3-yl)acetate (0.046 g, 0.095 mmol) in THF (2 mL) was added Ph₃P (0.050 g, 0.189 mmol) followes by (Z)-diethyl diazene-1,2-dicarboxylate (0.030 mL, 0.189 mmol) and the resulting mixture was stirred at rt for 16 h. Water (10 mL) was then added and the mixture was extracted with EtOAc, washed with brine (10 mL),dried (Na₂SO₄), filtered and concentrated. The residue was then purified via Biotage (4 g silica gel column, 0-50% EtOAc/hexane) to afford (2S)-isopropyl 2-(tert-butoxy)-2-(4-(4,4-dimethylpiperidin-1-yl)-5-(3-(2-(4-fluorophenoxy)ethyl)cyclobut-1-en-1-yl)-2,6-dimethylpyridin-3-yl)acetate (0.024 g, 0.041 mmol, 43.7% yield). LCMS (M+H): 581.7.

### EXAMPLE 25

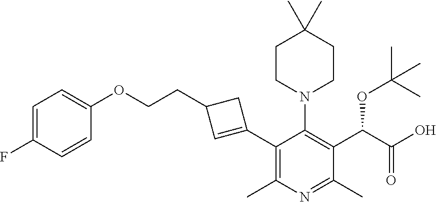 [SMILES: Cc1nc(C)c([C@H](OC(C)(C)C)C(=O)O)c(N2CCC(C)(C)CC2)c1C1=CC(CCOc2ccc(F)cc2)C1]

(2S)-2-(tert-Butoxy)-2-(4-(4,4-dimethylpiperidin-1-yl)-5-(3-(2-(4-fluorophenoxy)ethyl)cyclobut-1-en-1-yl)-2,6-dimethylpyridin-3-yl)acetic acid: A mixture of (2S)-isopropyl 2-(tert-butoxy)-2-(4-(4,4-dimethylpiperidin-1-yl)-5-(3-(2-(4-fluorophenoxy)ethyl)cyclobut-1-en-1-yl)-2,6-dimethylpyridin-3-yl)acetate (0.024 g, 0.041 mmol) and KOH (0.025 g, 0.446 mmol) in EtOH (1 mL) was stirrd at 82° C. for 18 h. The reaction mixture was filtered and purified by prep-HPLC to afford (2S)-2-(tert-butoxy)-2-(4-(4,4-dimethylpiperidin-1-yl)-5-(3-(2-(4-fluorophenoxy)ethyl)cyclobut-1-en-1-yl)-2,6-dimethylpyridin-3-yl)acetic acid (0.0105 g, 0.018 mmol, 44.3% yield). LCMS (M+H): 539.6.

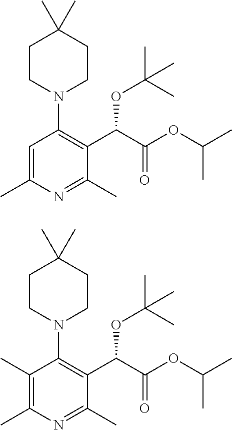

(S)-Isopropyl 2-(tert-butoxy)-2-(4-(4,4-dimethylpiperidin-1-yl)-2,6-dimethylpyridin-3-yl)acetate and (S)-isopropyl 2-(tert-butoxy)-2-(4-(4,4-dimethylpiperidin-1-yl)-2,5,6-trimethylpyridin-3-yl)acetate: A mixture of (S)-isopropyl 2-(5-bromo-4-(4,4-dimethylpiperidin-1-yl)-2,6-dimethylpyridin-3-yl)-2-(tert-butoxy)acetate (0.148 g, 0.315 mmol), methylboronic acid (0.057 g, 0.946 mmol) and 2M Na₂CO₃ (0.473 ml, 0.946 mmol) in DMF (3 mL) was degassed by bubbling N₂ through the reaction mixture for 10 min. Then, Pd(Ph₃P)₄ (0.036 g, 0.032 mmol) was added, degassed for 5 min and placed in pre-heated oil-bath at 100° C. After 3 h at 130° C., the reaction mixture was cooled and purified by prep-HPLC to give (S)-isopropyl 2-(tert-butoxy)-2-(4-(4,4-dimethylpiperidin-1-yl)-2,6-dimethylpyridin-3-yl)acetate. LCMS (M+H)=391.50. And (S)-isopropyl 2-(tert-butoxy)-2-(4-(4,4-dimethylpiperidin-1-yl)-2,5,6-trimethylpyridin-3-yl)acetate (0.0484 g, 0.120 mmol, 37.9% yield) as brown paste. ¹H NMR (500 MHz, CDCl₃) δ 6.21 (br. s., 1H), 5.04 (spt, J=6.3 Hz, 1H), 3.58-3.47 (m, 1H), 3.27-3.18 (m, 1H), 3.05 (d, J=11.5 Hz, 1H), 2.72 (d, J=11.7 Hz, 1H), 2.56 (s, 3H), 2.47 (s, 3H), 2.27 (s, 3H), 1.67-1.58 (m, 2H), 1.42 (d, J=12.5 Hz, 1H), 1.34 (d, J=15.1 Hz, 1H), 1.21 (d, J=6.1 Hz, 3H), 1.20 (s, 9H), 1.14 (d, J=6.1 Hz, 3H), 1.07 (s, 3H), 1.04 (s, 3H). LCMS (M+H)=405.55

### EXAMPLE 26

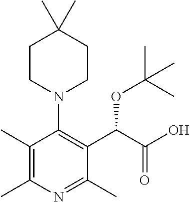 [SMILES: Cc1nc(C)c([C@H](OC(C)(C)C)C(=O)O)c(N2CCC(C)(C)CC2)c1C]

(S)-2-(tert-Butoxy)-2-(4-(4,4-dimethylpiperidin-1-yl)-2,5,6-trimethylpyridin-3-yl)acetic acid: To a solution of (S)-isopropyl 2-(tert-butoxy)-2-(4-(4,4-dimethylpiperidin-1-yl)-2,5,6-trimethylpyridin-3-yl)acetate (0.0456 g, 0.113 mmol) in 90% EtOH (2 mL) was added solid KOH (0.063 g, 1.127 mmol) and heated at reflux for 3.5 h. Then, cooled and purified by prep-HPLC to afford (S)-2-(tert-butoxy)-2-(4-(4,4-dimethylpiperidin-1-yl)-2,5,6-trimethylpyridin-3-yl)acetic acid (0.0341 g, 0.094 mmol, 83% yield) as solid. ¹H NMR (500 MHz, DMSO-d₆) δ 5.85 (br. s., 1H), 3.41 (br. s., 2H), 3.12 (br. s., 1H), 2.61 (d, J=12.8 Hz, 1H), 2.38 (s, 3H), 2.33 (s, 3H), 2.19 (s, 3H), 1.63-1.48 (m, 2H), 1.37 (d, J=11.7 Hz, 1H), 1.28 (d, J=12.5 Hz, 1H), 1.12 (s, 9H), 1.02 (br. s., 3H), 0.98 (br. s., 3H). LCMS (M +H)=363.2.

### EXAMPLE 27

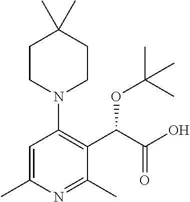 [SMILES: Cc1cc(N2CCC(C)(C)CC2)c([C@H](OC(C)(C)C)C(=O)O)c(C)n1]

(S)-2-(tert-Butoxy)-2-(4-(4,4-dimethylpiperidin-1-yl)-2,6-dimethylpyridin-3-yl)acetic acid: Hydrolysis of (S)-isopropyl 2-(tert-butoxy)-2-(4-(4,4-dimethylpiperidin-1-yl)-2,6-dimethylpyridin-3-yl)acetate according to the above procude provided (S)-2-(tert-butoxy)-2-(4-(4,4-dimethylpiperidin-1-yl)-2,6-dimethylpyridin-3-yl)acetic acid as white solid. ¹H NMR (500 MHz, CDCl₃) δ 6.77 (br. s., 1H), 5.50 (br. s., 1H), 3.54 (br. s., 2H), 2.87 (br. s., 2H), 2.68 (s, 3H), 2.55 (s, 3H), 1.68-1.60 (m, 2H), 1.58-1.50 (m, 2H), 1.21 (s, 9H), 1.06 (s, 6H). LCMS (M+H)=349.3.

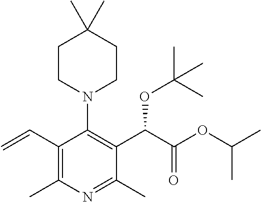 [SMILES: C=Cc1c(C)nc(C)c([C@H](OC(C)(C)C)C(=O)OC(C)C)c1N1CCC(C)(C)CC1]

(S)-Isopropyl 2-(tert-butoxy)-2-(4-(4,4-dimethylpiperidin-1-yl)-2,6-dimethyl-5-vinylpyridin-3-yl)acetate: A mixture of (S)-isopropyl 2-(5-bromo-4-(4,4-dimethylpiperidin-1-yl)-2,6-dimethylpyridin-3-yl)-2-(tert-butoxy)acetate (0.160 g, 0.341 mmol), 2-(4,6-divinyl-1,3,5,2,4,6-trioxatriborinan-2-yl)ethen-1-ylium, pyrrolidine salt (0.079 g, 0.341 mmol) and 2M Na₂CO₃ (0.511 ml, 1.022 mmol) in DMF (3 mL) was degassed by bubbling N₂ through the reaction mixture for 10 min. Then, Pd(Ph₃P)₄ (0.020 g, 0.017 mmol) was added, degassed for 5 min and placed in pre-heated oil-bath at 100° C. After 2 h at 120° C., the reaction mixture was cooled and purified by prep-HPLC to give (S)-isopropyl 2-(tert-butoxy)-2-(4-(4,4-dimethylpiperidin-1-yl)-2,6-dimethyl-5-vinylpyridin-3-yl)acetate (0.1112 g, 0.267 mmol, 78% yield) as viscous light brown oil. ¹H NMR (500 MHz, CDCl₃) δ 6.84 (dd, J=17.8, 11.2 Hz, 1H), 6.09 (br. s., 1H), 5.58 (dd, J=11.3, 2.0 Hz, 1H), 5.22 (dd, J=17.9, 2.0 Hz, 1H), 5.07 (spt, J=6.3 Hz, 1H), 3.61-3.49 (m, 1H), 3.16 (br. s., 1H), 3.04 (br. s., 1H), 2.57 (s, 3H), 2.53 (br. s., 1H), 2.46 (s, 3H), 1.65-1.50 (m, 2H), 1.43-1.26 (m, 2H), 1.22 (d, J=6.3 Hz, 3H), 1.19 (s, 10H), 1.01 (s, 6H). LCMS (M+H)=417.55.

### EXAMPLE 28

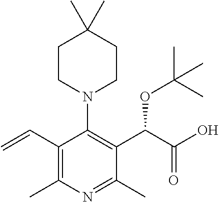 [SMILES: C=Cc1c(C)nc(C)c([C@H](OC(C)(C)C)C(=O)O)c1N1CCC(C)(C)CC1]

(S)-2-(tert-Butoxy)-2-(4-(4,4-dimethylpiperidin-1-yl)-2,6-dimethyl-5-vinylpyridin-3-yl)acetic acid: A mixture of (S)-isopropyl 2-(tert-butoxy)-2-(4-(4,4-dimethylpiperidin-1-yl)-2,6-dimethyl-5-vinylpyridin-3-yl)acetate (0.1062 g, 0.255 mmol) and solid KOH (0.143 g, 2.55 mmol) in 90% EtOH (3 mL) was refluxed for 4 h. Then, cooled and purified by prep-HPLC to afford (S)-2-(tert-butoxy)-2-(4-(4,4-dimethylpiperidin-1-yl)-2,6-dimethyl-5-vinylpyridin-3-yl)acetic acid (0.0755 g, 0.202 mmol, 79% yield) as pale yellow paste. ¹H NMR (500 MHz, DMSO-d₆) δ 6.88 (dd, J=17.8, 11.2 Hz, 1H), 5.76 (s, 1H), 5.56 (d, J=11.4 Hz, 1H), 5.17 (d, J=18.0 Hz, 1H), 3.51-3.29 (m, 2H), 2.95-2.85 (br.s., 1H), 3.44-3.37 (br.s.′ 1H), 2.39 (s, 3H), 2.31 (s, 3H), 1.61-1.42 (m, 2H), 1.39-1.20 (m, 2H), 1.10 (s, 9H), 0.96 (s, 6H). LCMS (M +H)=375.2.

### EXAMPLE 29

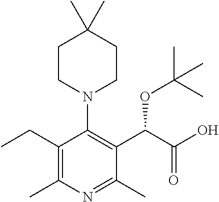 [SMILES: CCc1c(C)nc(C)c([C@H](OC(C)(C)C)C(=O)O)c1N1CCC(C)(C)CC1]

(S)-2-(tert-Butoxy)-2-(4-(4,4-dimethylpiperidin-1-yl)-5-ethyl-2,6-dimethylpyridin-3-yl)acetic acid: A mixture of (S)-2-(tert-butoxy)-2-(4-(4,4-dimethylpiperidin-1-yl)-2,6-dimethyl-5-vinylpyridin-3-yl)acetic acid (0.022 g, 0.059 mmol) and 10% Pd—C (6.25 mg, 5.87 μmol) in EtOH (5 mL) was evacuated and left under balloon hydrogen atmosphere for 3 h. Then, filtered through a plug of celite and concentrated to give (S)-2-(tert-butoxy)-2-(4-(4,4-dimethylpiperidin-1-yl)-5-ethyl-2,6-dimethylpyridin-3-yl)acetic acid (0.0155 g, 0.041 mmol, 70.1% yield) as white solid. ¹H NMR (500 MHz, CDCl₃) δ 5.86 (br. s., 1H), 3.48 (br. s., 2H), 3.01 (br. s., 2H), 2.82-2.75 (m, 1H), 2.73-2.66 (m, 1H), 2.64 (s, 3H), 2.56 (s, 3H), 1.68-1.43 (m, 4H), 1.25-1.20 (m, 13H), 1.05 (s, 6H). LCMS (M+H)=377.50.

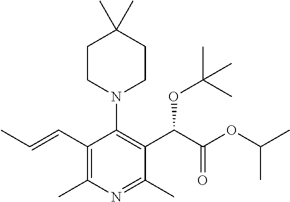 [SMILES: C/C=C/c1c(C)nc(C)c([C@H](OC(C)(C)C)C(=O)OC(C)C)c1N1CCC(C)(C)CC1]

(S,E)-Isopropyl 2-(tert-butoxy)-2-(4-(4,4-dimethylpiperidin-1-yl)-2,6-dimethyl-5-(prop-1-en-1-yl)pyridin-3-yl)acetate: A mixture of (S)-isopropyl 2-(5-bromo-4-(4,4-dimethylpiperidin-1-yl)-2,6-dimethylpyridin-3-yl)-2-(tert-butoxy)acetate (0.054 g, 0.115 mmol), (E)-prop-1-en-1-ylboronic acid (0.030 g, 0.345 mmol) and 2M Na₂CO₃ (0.173 ml, 0.345 mmol) in DMF (3 mL) was degassed by bubbling N₂ through the reaction mixture for 10 min. Then, Pd(Ph₃P)₄ (6.65 mg, 5.75 μmol) was added, degassed for 5 min and placed in pre-heated oil-bath at 100° C. After 3 h at 120° C., the reaction mixture was cooled, allowed to stir overnight (21 h) at rt and purified by prep-HPLC to give (S,E)-isopropyl 2-(tert-butoxy)-2-(4-(4,4-dimethylpiperidin-1-yl)-2,6-dimethyl-5-(prop-1-en-1-yl)pyridin-3-yl)acetate (0.0338 g, 0.078 mmol, 68.2% yield) as viscous pale yellow oil. ¹H NMR (500 MHz, CDCl₃) δ 6.44 (d, J=15.9 Hz, 1H), 6.10 (br. s., 1H), 5.57 (dq, J=15.9, 6.5 Hz, 1H), 5.10-5.02 (m, 1H), 3.58-3.44 (m, 1H), 3.13 (br. s., 1H), 3.03 (d, J=11.2 Hz, 1H), 2.56 (s, 3H), 2.50 (d, J=7.9 Hz, 1H), 2.43 (s, 3H), 1.95 (dd, J=6.6, 1.7 Hz, 3H), 1.56 (br. s., 2H), 1.42-1.34 (m, 1H), 1.33-1.26 (m, 1H), 1.22 (d, J=6.3 Hz, 3H), 1.19 (s, 9H), 1.18 (d, J=6.3 Hz, 3H), 1.01 (s, 6H). LCMS (M+H)=431.55.

### EXAMPLE 30

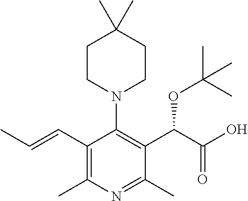 [SMILES: C/C=C/c1c(C)nc(C)c([C@H](OC(C)(C)C)C(=O)O)c1N1CCC(C)(C)CC1]

(S,E)-2-(tert-Butoxy)-2-(4-(4,4-dimethylpiperidin-1-yl)-2,6-dimethyl-5-(prop-1-en-1-yl)pyridin-3-yl)acetic acid: A mixture of (S,E)-isopropyl 2-(tert-butoxy)-2-(4-(4,4-dimethylpiperidin-1-yl)-2,6-dimethyl-5-(prop-1-en-1-yl)pyridin-3-yl)acetate (0.0315 g, 0.073 mmol) and solid KOH (0.041 g, 0.731 mmol) in 90% EtOH (3 mL) was refluxed for 6 h. Then, cooled and purified by prep-HPLC to afford (S,E)-2-(tert-butoxy)-2-(4-(4,4-dimethylpiperidin-1-yl)-2,6-dimethyl-5-(prop-1-en-1-yl)pyridin-3-yl)acetic acid (0.0255 g, 0.066 mmol, 90% yield) as solid. ¹H NMR (500 MHz, DMSO-d₆) δ 6.47 (d, J=16.1 Hz, 1H), 5.86 (s, 1H), 5.58-5.47 (m, 1H), 3.40 (br.s., 2H), 2.93 (br. s., 1H), 2.43-2.38 (m, 1H), 2.38 (s, 3H), 2.29 (s, 3H), 1.89 (d, J=6.6 Hz, 3H), 1.61-1.41 (m, 2H), 1.39-1.21 (m, 2H), 1.11 (s, 9H), 0.96 (s, 6H). LCMS (M+H)=389.20.

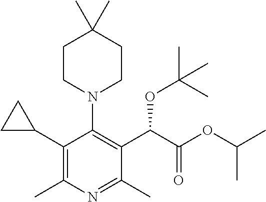 [SMILES: Cc1nc(C)c([C@H](OC(C)(C)C)C(=O)OC(C)C)c(N2CCC(C)(C)CC2)c1C1CC1]

(S)-Isopropyl 2-(tert-butoxy)-2-(5-cyclopropyl-4-(4,4-dimethylpiperidin-1-yl)-2,6-dimethylpyridin-3-yl)acetate: A mixture of (S)-isopropyl 2-(5-bromo-4-(4,4-dimethylpiperidin-1-yl)-2,6-dimethylpyridin-3-yl)-2-(tert-butoxy)acetate (0.315 g, 0.671 mmol), cyclopropylboronic acid (0.1 g, 1.164 mmol) and 2M Na₂CO₃ (0.671 ml, 1.342 mmol) in DMF (5 mL) was degassed for 10 min by bubbling N2 through the reaction mixture. Then, Pd(Ph₃P)₄ (0.078 g, 0.067 mmol) was added, degassed for 5 min and placed in a pre-heated oil bath at 100° C. After h at 1 C, cooled and purified by prep-HPLC to afford (S)-isopropyl 2-(tert-butoxy)-2-(5-cyclopropyl-4-(4,4-dimethylpiperidin-1-yl)-2,6-dimethylpyridin-3-yl)acetate (0.0224 g, 0.052 mmol, 7.75% yield) as pale yellow paste. LCMS (M+H)=431.55.

### EXAMPLE 31

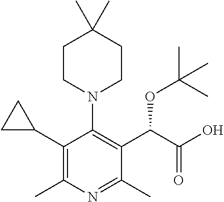 [SMILES: Cc1nc(C)c([C@H](OC(C)(C)C)C(=O)O)c(N2CCC(C)(C)CC2)c1C1CC1]

(S)-2-(tert-Butoxy)-2-(5-cyclopropyl-4-(4,4-dimethylpiperidin-1-yl)-2,6-dimethylpyridin-3-yl)acetic acid: To a solution of (S)-isopropyl 2-(tert-butoxy)-2-(5-cyclopropyl-4-(4,4-dimethylpiperidin-1-yl)-2,6-dimethylpyridin-3-yl)acetate (0.022 g, 0.051 mmol) in EtOH (2 mL) was added solid KOH (0.029 g, 0.511 mmol) and refluxed for h. Then, cooled and purified by prep-HPLC to afford (S)-2-(tert-butoxy)-2-(5-cyclopropyl-4-(4,4-dimethylpiperidin-1-yl)-2,6-dimethylpyridin-3-yl)acetic acid (0.0186 g, 0.048 mmol, 94% yield). LCMS (M+H)=389.2.

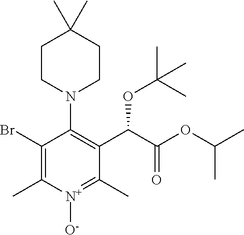 [SMILES: Cc1c(Br)c(N2CCC(C)(C)CC2)c([C@H](OC(C)(C)C)C(=O)OC(C)C)c(C)[n+]1[O-]]

(S)-3-Bromo-5-(1-(tert-butoxy)-2-isopropoxy-2-oxoethyl)-4-(4,4-dimethylpiperidin-1-yl)-2,6-dimethylpyridine 1-oxide: To a stirred solution of (S)-isopropyl 2-(5-bromo-4-(4,4-dimethylpiperidin-1-yl)-2,6-dimethylpyridin-3-yl)-2-(tert-butoxy)acetate (16 g, 34 mmol) in DCM (170 ml) was added 77% mCPBA (11.7 g, 51.1 mmol) at rt over 5 min. After 4 h, the reaction mixture was washed with sat.aq Na₂CO₃ (3×50 mL), dried (Na₂SO₄), filtered and concentrated to give (S)-3-bromo-5-(1-(tert-butoxy)-2-isopropoxy-2-oxoethyl)-4-(4,4-dimethylpiperidin-1-yl)-2,6-dimethylpyridine 1-oxide (14.6 g, 30.1 mmol, 88% yield). ¹H NMR (500 MHz, CDCl₃) δ 6.28 (br. s., 1H), 5.03 (spt, J=6.3 Hz, 1H), 4.00 (t, J=11.4 Hz, 1H), 3.50 (td, J=12.1, 2.4 Hz, 1H), 2.91-2.79 (m, 1H), 2.76 (s, 3H), 2.67-2.60 (m, 1H), 2.56 (s, 3H), 1.60 (br. s., 1H), 1.45 (d, J=12.1 Hz, 1H), 1.38-1.31 (m, 1H), 1.22-1.17 (m, 13H), 1.14 (d, J=6.1 Hz, 3H), 1.10-1.05 (m, 3H), 1.04-1.00 (m, 3H). LCMS (M+)=485.10, 487.10.

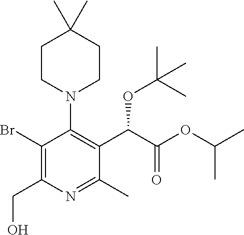 [SMILES: Cc1nc(CO)c(Br)c(N2CCC(C)(C)CC2)c1[C@H](OC(C)(C)C)C(=O)OC(C)C]

Isopropyl (S)-2-(5-bromo-4-(4,4-dimethylpiperidin-1-yl)-6-(hydroxymethyl)-2-methylpyridin-3-yl)-2-(tert-butoxy)acetate: To a stirred solution of (S)-3-bromo-5-(1-(tert-butoxy)-2-isopropoxy-2-oxoethyl)-4-(4,4-dimethylpiperidin-1-yl)-2,6-dimethylpyridine 1-oxide (12.8 g, 26.4 mmol) in anhydrous DCM (132 ml) was added, dropwise, trifluoroacetic anhydride (7.45 ml, 52.7 mmol) over 5 min at rt. After 2 h, sat NaHCO₃ (50 mL) was slowly added, stirred for 10 min, aq layer separated, organic layer dried (Na₂SO₄), filtered, concentrated, adsorbed onto celite and was purified on silica gel (Biotage, EtOAc/hexanes gradient). The major peak was collected to afford (S)-isopropyl 2-(5-bromo-4-(4,4-dimethylpiperidin-1-yl)-6-(hydroxymethyl)-2-methylpyridin-3-yl)-2-(tert-butoxy)acetate (9.7 g, 20 mmol, 76% yield). ¹H NMR (500 MHz, CDCl₃) δ 6.24 (br. s., 1H), 5.04 (spt, J=6.3 Hz, 1H), 4.75 (br. s., 1H), 4.72-4.59 (m, 2H), 4.05 (br. s., 1H), 3.48 (t, J=11.0 Hz, 1H), 2.91 (d, J=11.5 Hz, 1H), 2.68-2.62 (m, 1H), 2.60 (s, 3H), 1.63-1.57 (m, 2H), 1.45 (d, J=15.0 Hz, 1H), 1.39-1.32 (m, 1H), 1.22-1.19 (m, 12H), 1.15-1.12 (m, 3H), 1.08 (s, 3H), 1.03 (s, 3H). LCMS (M+H)=485.17, 487.17.

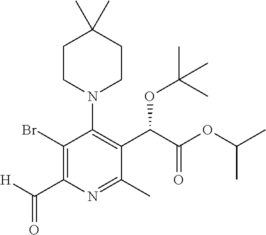 [SMILES: Cc1nc(C=O)c(Br)c(N2CCC(C)(C)CC2)c1[C@H](OC(C)(C)C)C(=O)OC(C)C]

Isopropyl (S)-2-(5-bromo-4-(4,4-dimethylpiperidin-1-yl)-6-formyl-2-methylpyridin-3-yl)-2-(tert-butoxy)acetate: To a stirred solution of (S)-isopropyl 2-(5-bromo-4-(4,4-dimethylpiperidin-1-yl)-6-(hydroxymethyl)-2-methylpyridin-3-yl)-2-(tert-butoxy)acetate (1.0 g, 2.1 mmol) in CH₂Cl₂ (19 ml) was added Dess-Martin Periodinane (1.3 g, 3.1 mmol) at once at rt. After 16 h, the reaction mixture was diluted with ether, washed with 1M NaOH followed by brine. The organic phase was dried over (Na₂SO₄), concentrated and purified on silica gel (Biotage, EtOAc/hexanes gradient, 0-100% over 10 CVs) to afford (S)-isopropyl 2-(5-bromo-4-(4,4-dimethylpiperidin-1-yl)-6-formyl-2-methylpyridin-3-yl)-2-(tert-butoxy)acetate (960 mg, 1.99 mmol, 96% yield). ¹H NMR (500 MHz, CDCl₃) δ 10.29 (s, 1H), 6.26 (br. s., 1H), 5.12-4.97 (m, 1H), 4.15-4.05 (m, 1H), 3.54 (t, J=12.1 Hz, 1H), 2.94 (d, J=10.9 Hz, 1H), 2.71 (d, J=11.0 Hz, 1H), 2.66-2.62 (m, 3H), 1.59 (br. s., 1H), 1.51 (br. s., 1H), 1.41-1.35 (m, 1H), 1.30-1.25 (m, 1H), 1.22-1.18 (m, 12H), 1.16-1.13 (m, 3H), 1.11-1.03 (m, 6H). LCMS (M+H)=483.0, 485.0.

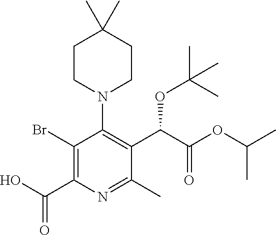 [SMILES: Cc1nc(C(=O)O)c(Br)c(N2CCC(C)(C)CC2)c1[C@H](OC(C)(C)C)C(=O)OC(C)C]

(S)-3-Bromo-5-(1-(tert-butoxy)-2-isopropoxy-2-oxoethyl)-4-(4,4-dimethylpiperidin-1-yl)-6-methylpicolinic acid: To a solution of (S)-isopropyl 2-(5-bromo-4-(4,4-dimethylpiperidin-1-yl)-6-formyl-2-methylpyridin-3-yl)-2-(tert-butoxy)acetate (2.0 g, 4.1 mmol) in DMSO (41 ml) was added potassium phosphate monobasic (1.69 g, 12.4 mmol) in water (10 mL) followed by sodium chlorite (1.12 g, 12.4 mmol) in water (10 mL) and the mixture was stirred overnight. A precipitate formed immediately. As the reaction stirred, precipitate came out of the solution and stuck to the sides of the flask. After stirring overnight, the solution was poured away and the solids were taken up in EtOAc and was then washed with brine, dried (Na₂SO₄), filtered and concentrated to afford the expected product. The mother liquor also contained some product. It was diluted with EtOAc and washed with brine. The organic phase was dried over Na₂SO₄, and concentrated and was combined with the material isolated from the precipitate. The combined material afforded a quantitative amount of (S)-3-bromo-5-(1-(tert-butoxy)-2-isopropoxy-2-oxoethyl)-4-(4,4-dimethylpiperidin-1-yl)-6-methylpicolinic acid (quantitative). LCMS (M+H)=499.04.

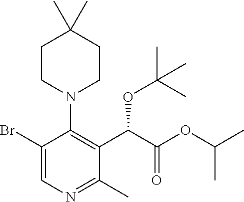 [SMILES: Cc1ncc(Br)c(N2CCC(C)(C)CC2)c1[C@H](OC(C)(C)C)C(=O)OC(C)C]

Isopropyl (S)-2-(5-bromo-4-(4,4-dimethylpiperidin-1-yl)-2-methylpyridin-3-yl)-2-(tert-butoxy)acetate: Water (0.16 ml, 8.8 mmol) followed by diphenylphosphoryl azide (0.76 ml, 3.5 mmol) was added to a stirring solution of (S)-3-bromo-5-(1-(tert-butoxy)-2-isopropoxy-2-oxoethyl)-4-(4,4-dimethylpiperidin-1-yl)-6-methylpicolinic acid (882 mg, 1.77 mmol) in toluene (18 ml) at rt. The reaction was stirred at 90° C. for 2 hrs. The mixture was then diluted with EtOAc and washed with sat aq NaHCO₃. The organic phase was dried over Na₂SO₄, filtered and concentrated. The reaction was concentrated, adsorbed onto celite and was purified on silica gel (Biotage, EtOAc/hexanes gradient, 0-100% over 10 CVs) to give the expected product (S)-isopropyl 2-(5-bromo-4-(4,4-dimethylpiperidin-1-yl)-2-methylpyridin-3-yl)-2-(tert-butoxy)acetate in quantiative isolated yield. LCMS (M+H)=455.20, 457.20.

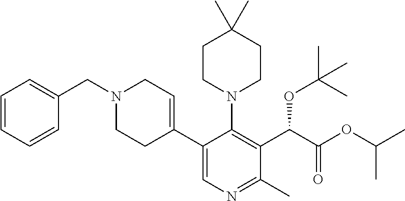 [SMILES: Cc1ncc(C2=CCN(Cc3ccccc3)CC2)c(N2CCC(C)(C)CC2)c1[C@H](OC(C)(C)C)C(=O)OC(C)C]

Isopropyl (S)-2-(1′-benzyl-4-(4,4-dimethylpiperidin-1-yl)-6-methyl-1′,2′,3′,6′-tetrahydro-[3,4′-bipyridin]-5-yl)-2-(tert-butoxy)acetate : (S)-Isopropyl 2-(5-bromo-4-(4,4-dimethylpiperidin-1-yl)-2-methylpyridin-3-yl)-2-(tert-butoxy)acetate (100 mg, 0.220 mmol), 1-benzyl-4-(4,4,5,5-tetramethyl-1,3,2-dioxaborolan-2-yl)-1,2,3,6-tetrahydropyridine hydrochloride (81 mg, 0.24 mmol), 2-dicyclohexylphosphino-2′,6′-dimethoxybiphenyl (18 mg, 0.044 mmol), Pd(OAc)₂ (5 mg, 0.022 mmol) and potassium phosphate tribasic (350 mg, 1.65 mmol) were combined under N₂. 1,4-Dioxane (3.7 ml) and Water (0.7 ml) was added under N₂. The reaction was heated at 80° C. for 1 hr. The reaction was concentrated, adsorbed onto celite and was purified on silica gel (Biotage, EtOAc/hexanes gradient 0-100% over 10CVs) to give the desired product (S)-isopropyl 2-(1′-benzyl-4-(4,4-dimethylpiperidin-1-yl)-6-methyl-1′,2′,3′,6′-tetrahydro-[3,4′-bipyridin]-5-yl)-2-(tert-butoxy)acetate (55 mg, 0.10 mmol, 46% yield). LCMS (M+H)=548.35.

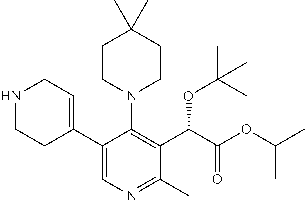 [SMILES: Cc1ncc(C2=CCNCC2)c(N2CCC(C)(C)CC2)c1[C@H](OC(C)(C)C)C(=O)OC(C)C]

Isopropyl (S)-2-(tert-butoxy)-2-(4-(4,4-dimethylpiperidin-1-yl)-6-methyl-1′,2′,3′,6′-tetrahydro-[3,4′-bipyridin]-5-yl)acetate: Under an atmosphere of N₂, 10% Pd—C (78 mg, 0.073 mmol) was added to a stirring solution of (S)-isopropyl 2-(1′-benzyl-4-(4,4-dimethylpiperidin-1-yl)-6-methyl-1′,2′,3′,6′-tetrahydro-[3,4′-bipyridin]-5-yl)-2-(tert-butoxy)acetate (200 mg, 0.365 mmol) in ethanol (3.6 ml) at rt. The atmosphere was replaced with H₂ (g) and the reaction was stirred overnight under a balloon of H₂ (g). The atmosphere was then replaced with N₂ and the reaction was filtered through a pad of celite, washing with EtOAc. The filtrate was concentrated to give the product (S)-isopropyl 2-(tert-butoxy)-2-(4-(4,4-dimethylpiperidin-1-yl)-6-methyl-1′,2′,3′,6′-tetrahydro-[3,4′-bipyridin]-5-yl)acetate (80 mg, 0.18 mmol, 48% yield). LCMS (M+H)=458.25.

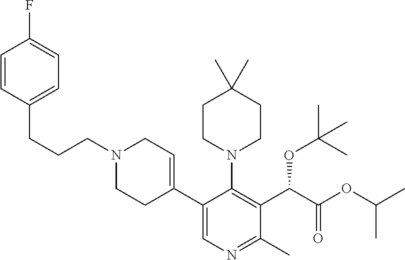 [SMILES: Cc1ncc(C2=CCN(CCCc3ccc(F)cc3)CC2)c(N2CCC(C)(C)CC2)c1[C@H](OC(C)(C)C)C(=O)OC(C)C]

(S)-Isopropyl 2-(tert-butoxy)-2-(4-(4,4-dimethylpiperidin-1-yl)-1′-(3-(4-fluorophenyl)propyl)-6-methyl-1′,2′,3′,6′-tetrahydro-[3,4′-bipyridin]-5-yl)acetate: To a stirred solution of (S)-isopropyl 2-(tert-butoxy)-2-(4-(4,4-dimethylpiperidin-1-yl)-6-methyl-1′,2′,3′,6′-tetrahydro-[3,4′-bipyridin]-5-yl)acetate (80 mg, 0.18 mmol) and 3-(4-fluorophenyl)propanal (53 mg, 0.35 mmol) in MeOH (2 mL) was added at once NaCNBH₄ (16 mg, 0.26 mmol) and ZnCl₂ (18 mg, 0.13 mmol) at rt and the mixture was stirred for 2 h. At this point LCMS indicates completion of reaction. The reaction was concentrated, adsorbed onto celite and was purified on silica gel (Biotage, EtOAc/hexanes gradient, 0-100% over 10 CVs). The product didn't elute with EtOAc/hex, therefore the column was flushed with 10% MeoH/DCM and the product was isolated to provide (S)-isopropyl 2-(tert-butoxy)-2-(4-(4,4-dimethylpiperidin-1-yl)-1′-(3-(4-fluorophenyl)propyl)-6-methyl-1′,2′,3′,6′-tetrahydro-[3,4′-bipyridin]-5-yl)acetate (72 mg, 0.12 mmol, 69% yield). LCMS (M+H)=594.4.

### EXAMPLE 32

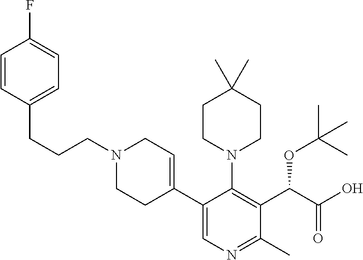 [SMILES: Cc1ncc(C2=CCN(CCCc3ccc(F)cc3)CC2)c(N2CCC(C)(C)CC2)c1[C@H](OC(C)(C)C)C(=O)O]

(S)-2-(tert-Butoxy)-2-(4-(4,4-dimethylpiperidin-1-yl)-1′-(3-(4-fluorophenyl)propyl)-6-methyl-1′,2′,3′,6′-tetrahydro-[3,4′-bipyridin]-5-yl)acetic acid: 5N aq.NaOH (0.1 ml, 0.6 mmol) was added to a stirring solution of (S)-isopropyl 2-(tert-butoxy)-2-(4-(4,4-dimethylpiperidin-1-yl)-1′-(3-(4-fluorophenyl)propyl)-6-methyl-1′,2′,3′,6′-tetrahydro-[3,4′-bipyridin]-5-yl)acetate (36 mg, 0.06 mmol) in EtOH (0.6 ml) at 80° C. The reaction was allowed to stir overnight at 80° C. and then was purified by preparative reverse phase HPLC on a C18 column using a suitably buffered H₂O/CH₃CN gradient, and concentrated by lyophilization to give the expected product (S)-2-(tert-butoxy)-2-(4-(4,4-dimethylpiperidin-1-yl)-1′-(3-(4-fluorophenyl)propyl)-6-methyl-1′,2′,3′,6′-tetrahydro-[3,4′-bipyridin]-5-yl)acetic acid (20 mg, 0.034 mmol, 57% yield) consistent by LCMS and NMR. ¹H NMR (500 MHz, Methanol-d₄) δ 7.90 (s, 1H), 7.25 (dd, J=8.7, 5.4 Hz, 2H), 7.05-6.99 (m, 2H), 5.71 (br. s., 1H), 5.61 (s, 1H), 3.66 (br. s., 2H), 3.57-3.43 (m, 2H), 3.11 (br. s., 2H), 2.93-2.81 (m, 4H), 2.71 (t, J=7.6 Hz, 2H), 2.62 (d, J=19.1 Hz, 1H), 2.57 (s, 3H), 2.46 (d, J=19.1 Hz, 1H), 2.04-1.97 (m, 2H), 1.60 (br. s., 2H), 1.49 (br. s., 2H), 1.16-1.13 (m, 9H), 1.02 (s, 6H). LCMS (M+H)=552.4.

### Biological Methods

Inhibition of HIV replication: A recombinant NL-RLuc proviral clone was constructed in which a section of the nef gene from NL4-3 was replaced with the Renilla Luciferase gene. This virus is fully infectious and can undergo multiple cycles of replication in cell culture. In addition, the luciferous reporter provides a simple and easy method for quantitating the extent of virus growth and consequently, the antiviral activity of test compounds. The plasmid pNLRLuc contains the proviral NL-Rluc DNA cloned into pUC18 at the Pvull site. The NL-RLuc virus was prepared by transfection of 293T cells with the plasmid pNLRLuc. Transfections were performed using the LipofectAMINE PLUS kit from Invitrogen (Carlsbad, Calif.) according to the manufacturer and the virus generated was titered in MT-2 cells. For susceptibility analyses, the titrated virus was used to infect MT-2 cells in the presence of compound, and after 5 days of incubation, cells were processed and quantitated for virus growth by the amount of expressed luciferase. Assay media was RPMI 1640 supplemented with 10% heat inactivated fetal bovine serum (FBS), 100 units/ml penicillin G/100 units/ml streptomycin, 10 mM HEPES buffer pH 7.55 and 2 mM L-glutamine. The results from at least 2 experiments were used to calculate the EC₅₀ values. Luciferase was quantitated using the Dual Luciferase kit from Promega (Madison, Wis.). Susceptibility of viruses to compounds was determined by incubation in the presence of serial dilutions of the compound. The 50% effective concentration (EC₅₀) was calculated by using the exponential form of the median effect equation where (Fa)=1/[1+(ED₅₀/drug conc.)^{m}] (Johnson V A, Byington R T. Infectivity Assay. In Techniques in HIV Research. ed. Aldovini A, Walker BD. 71-76. New York: Stockton Press.1990). Results are shown in Table 1. Activity equal to A refers to a compound having an EC₅₀<100 nM, while B and C denote compounds having an EC₅₀ between 100 nM and 1 uM (B) or >1 uM (C).

TABLE 1

| Example | Activity | EC₅₀ μM |
|---|---|---|
| 1 | B | 0.282 |
| 2 | A | 0.064 |
| 3 | A | |
| 4 | A | |
| 5 | A | 0.035 |
| 6 | A | |
| 7 | A | |
| 8 | B | |
| 9 | B | 0.281 |
| 10 | B | |
| 11 | A | 0.027 |
| 12 | B | |
| 13 | B | |
| 14 | B | 0.136 |
| 15 | B | |
| 16 | A | |
| 17 | A | |
| 18 | B | |
| 19 | A | |
| 20 | B | |
| 21 | C | 2.213 |
| 22 | B | |
| 23 | A | |
| 24 | B | |
| 25 | A | |
| 26 | A | 0.097 |
| 27 | C | |
| 28 | B | |
| 29 | A | 0.068 |
| 30 | B | |
| 31 | B | 0.118 |
| 32 | A | 0.002 |

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

1. A compound of Formula I or a pharmaceutically acceptable salt thereof 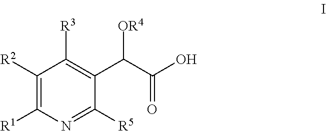
wherein:
R¹ is selected from hydrogen or alkyl;
R² is selected from hydrogen, halo, cyano, alkyl, (R⁶)alkyl, alkenyl, (R⁶)alkenyl, alkynyl, (R⁶)alkynyl, cycloalkyl, (alkyl)cycloalkyl, cycloalkenyl, (alkyl)cycloalkenyl, (R⁶)cycloalkenyl, (R⁷)NHCH₂CH═CH—, (R⁷)tetrahydropyridinyl, or ((N-benzyl-4-hydroxy)piperidin-4-yl)ethynyl;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, alkoxy, and haloalkoxy;
R⁴ is selected from alkyl or haloalkyl;
R⁵ is alkyl;
R⁶ is selected from Ar¹, (Ar¹)alkyl, (Ar¹O)alkyl or benzyloxy,
R⁷ is selected from hydrogen, (Ar¹)alkyl, alkoxycarbonyl, or benzyloxycarbonyl; and
Ar¹ is phenyl substituted with 0-3 substituents selected from halo, alkyl, haloalkyl, alkoxy, haloalkoxy, or phenyl.

2. A compound or salt of claim 1 wherein R³ is piperidinyl substituted with 0-3 substituents selected from cyano, halo, alkyl, haloalkyl, alkoxy, or haloalkoxy.

3. A compound or salt of claim 1 where R² is selected from alkyl, (R⁶)alkyl, alkenyl, (R⁶)alkenyl, alkynyl, or (R⁶)alkynyl.

4. A compound or salt of claim 1 where R² is selected from cycloalkyl, (alkyl)cycloalkyl, cycloalkenyl, (alkyl)cycloalkenyl, or (R⁶)cycloalkenyl.

5. A compound or salt of claim 1 where R² is (R⁷)NHCH₂CH═CH— or (R⁷)tetrahydropyridinyl.

6. A compound of Formula I or a pharmaceutically acceptable salt thereof 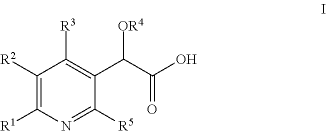
wherein:
R¹ is selected from hydrogen or alkyl;
R² is selected from hydrogen, halo, cyano, alkyl, (R⁶)alkyl, alkenyl, (R⁶)alkenyl, alkynyl, (R⁶)alkynyl, cycloalkyl, (alkyl)cycloalkyl, cycloalkenyl, (alkyl)cycloalkenyl, (R⁶)cycloalkenyl, (R⁷)NHCH₂CH═CH—, (R⁷)tetrahydropyridinyl, or ((N-benzyl-4-hydroxy)piperidin-4-yl)ethynyl;
R³ is piperidinyl substituted with 0-3 substituents selected from cyano, halo, alkyl, haloalkyl, alkoxy, or haloalkoxy
R⁴ is selected from alkyl or haloalkyl;
R⁵ is alkyl;
R⁶ is selected from Ar¹, (Ar¹)alkyl, (Ar¹O)alkyl or benzyloxy,
R⁷ is selected from hydrogen, (Ar¹)alkyl, alkoxycarbonyl, or benzyloxycarbonyl; and
Ar¹ is phenyl substituted with 0-3 substituents selected from halo, alkyl, haloalkyl, alkoxy, haloalkoxy, or phenyl.

7. A compound of Formula I or a pharmaceutically acceptable salt thereof 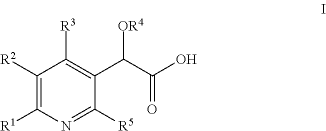
wherein:
R¹ is selected from hydrogen or alkyl;
R² is selected from alkyl, (R⁶)alkyl, alkenyl, (R⁶)alkenyl, alkynyl, or (R⁶)alkynyl;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, alkoxy, and haloalkoxy;
R⁴ is selected from alkyl or haloalkyl;
R⁵ is alkyl;
R⁶ is selected from Ar¹, (Ar¹)alkyl, (Ar¹O)alkyl or benzyloxy,
R⁷ is selected from hydrogen, (Ar¹)alkyl, alkoxycarbonyl, or benzyloxycarbonyl; and
Ar¹ is phenyl substituted with 0-3 substituents selected from halo, alkyl, haloalkyl, alkoxy, haloalkoxy, or phenyl; or a pharmaceutically acceptable salt thereof.

8. A compound of Formula I or a pharmaceutically acceptable salt thereof 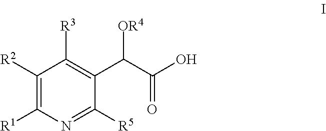
wherein:
R¹ is selected from hydrogen or alkyl;
R² is selected from cycloalkyl, (alkyl)cycloalkyl, cycloalkenyl, (alkyl)cycloalkenyl, or (R⁶)cycloalkenyl;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, alkoxy, and haloalkoxy;
R⁴ is selected from alkyl or haloalkyl;
R⁵ is alkyl;
R⁶ is selected from Ar¹, (Ar¹)alkyl, (Ar¹O)alkyl or benzyloxy,
R⁷ is selected from hydrogen, (Ar¹)alkyl, alkoxycarbonyl, or benzyloxycarbonyl; and
Ar¹ is phenyl substituted with 0-3 substituents selected from halo, alkyl, haloalkyl, alkoxy, haloalkoxy, or phenyl.

9. A compound of Formula I or a pharmaceutically acceptable salt thereof 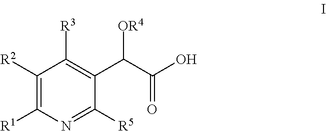
wherein:
R¹ is selected from hydrogen or alkyl;
R² is selected from (R⁷)NHCH₂CH═CH— or (R⁷)tetrahydropyridinyl;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, alkoxy, and haloalkoxy;
R⁴ is selected from alkyl or haloalkyl;
R⁵ is alkyl;
R⁶ is selected from Ar¹, (Ar¹)alkyl, (Ar¹O)alkyl or benzyloxy,
R⁷ is selected from hydrogen, (Ar¹)alkyl, alkoxycarbonyl, or benzyloxycarbonyl; and
Ar¹ is phenyl substituted with 0-3 substituents selected from halo, alkyl, haloalkyl, alkoxy, haloalkoxy, or phenyl.

10. A composition useful for treating HIV infection comprising a compound of claim 1 and a pharmaceutically acceptable carrier.

11. The composition of claim 10 further comprising at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

12. The composition of claim 11 wherein the other agent is dolutegravir.

13. A method for treating HIV infection comprising administering a compound of claim 1, or a pharmaceutically acceptable salt thereof, to a patient in need thereof.

14. The method of claim 13 further comprising administering at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

15. The method of claim 14 wherein the other agent is dolutegravir.

16. The method of claim 14 wherein the other agent is administered to the patient prior to, simultaneously with, or subsequently to the compound of claim 1.
